(19) United States
(12) Patent Application Publication
(10) Pub. No.: US 2018/0087070 A1
(43) Pub. Date: Mar. 29, 2018

(54) ENHANCED BACULOVIRUS YIELD

(71) Applicant: THE UNIVERSITY OF QUEENSLAND, St Lucia (AU)

(72) Inventors: Leila MATINDOOST, St Lucia (AU); Sassan ASGARI, St Lucia (AU); Steven REID, St Lucia (AU)

(21) Appl. No.: 15/563,408

(22) PCT Filed: Mar. 31, 2016
(86) PCT No.: PCT/AU2016/050243

§ 371 Date: Sep. 29, 2017

(30) Foreign Application Priority Data
Mar. 31, 2015 (AU) 2015901162

(51) Int. Cl. C12N 15/866; C07K 14/01

(52) U.S. Cl. CPC C12N 15/866; C07K 14/01; C12N 2710/14052; C12N 2710/14043; C12N 2710/14022

(57) ABSTRACT

A method for increasing or enhancing baculovirus yield and/or recombinant protein expression from a baculovirus is provided, wherein said method includes the step of engineering said host cell to express one or more of a p35, p35-trunc, p35-trunc-tail, and p49 protein. In some embodiments, said baculovirus does not normally encode a p35 protein, and said method includes the step of engineering said host cell to express one or more of a p35, p35-trunc, and p35-trunc-tail protein. In some embodiments, said baculovirus does not normally encode a p49 protein, and said method includes the step of engineering said host cell to express a p49 protein. Also provided are isolated p35-trunc and p35-trunc-tail proteins.

### TECHNICAL FIELD

THE present invention relates to baculoviruses and host cells infected by baculoviruses. More particularly, the invention relates to methods of increasing baculovirus yield and/or recombinant protein expression in host cells.

### BACKGROUND

Baculoviruses are a family of large rod-shaped viruses that infect arthropods (especially insects). Baculoviruses have been used for almost a century worldwide as insecticides (Inceoglu et al. 2006) and have significant advantages compared to chemical insecticides; baculoviruses are natural insect pathogens, highly specific to insects, and pathogenically safe to vertebrates and other beneficial organisms. In particular, Helicoverpa and Heliothis species are key pests worldwide (Moscardi et al., 2011) that can be controlled by the use of baculovirus biopesticides.

Commercial scale production of baculoviruses is currently performed exclusively in vivo, generally by growing larvae in the laboratory using feed contaminated with baculovirus. This has significantly inhibited the commercial use of baculovirus biopesticides, with difficulties in scaling up commercial in vivo production due to significant labour costs. Production of baculoviruses in vitro is considered to have many potential advantages, including providing a more reliable manufacturing base for baculovirus biopesticides. However, commercial-scale in vitro production of baculovirus biopesticides is not considered viable at current yields.

In addition to their use as biopesticides, baculoviruses have application for the production of recombinant proteins. In particular, recombinant Autographa californica multiple nucleopolyhedrovirus (“AcMNPV”) is commonly exploited for recombinant protein expression in host cells. Improving the yield of recombinant proteins expressed from baculoviruses has great potential for commercial-scale production of recombinant proteins, e.g. vaccines.

Baculovirus genomes contain some species-specific genes, or genes specific to certain baculovirus lineages, as well as groups of conserved genes in the family. Moreover, in the course of evolution, baculovirus genomes have been subjected to a high level of gene losses and gene acquisitions from their hosts (Herniou et al. 2003). Of these adapted genes, p35 and inhibitor of apoptosis (IAP) proteins play important roles in baculovirus-host interaction by inhibition of the host cell apoptosis (Clem 2007). Additionally, certain viruses possess viral suppressors of RNAi (VSRs), which can provide a counter-defence to the production of antiviral RNAi by host cells. However, VSRs have not previously been identified in baculoviruses.

### SUMMARY

The present invention addresses the need for increased yields of baculoviruses in host cells. It is also an objective of the invention to provide increased expression of recombinant proteins in host cells using recombinant baculoviruses.

One broad aspect of the invention therefore relates to increasing baculovirus yield in a host cell, by engineering said host cell to express one or more proteins, to thereby increase baculovirus yield in the host cell. In some embodiments, increased or enhanced baculovirus yield may facilitate increased or enhanced recombinant protein expression by the baculovirus.

In a first broad form, the invention provides a method for increasing or enhancing baculovirus yield and/or recombinant protein expression from a baculovirus in a host cell, said method including the step of engineering said host cell to express one or more of the proteins comprising the amino acid sequences set forth in SEQ ID NOS:1-4, respectively, or fragments or variants thereof, to thereby increase or enhance baculovirus yield and/or recombinant protein expression from the baculovirus in the host cell.

In a first aspect of the first broad form, the invention provides a method for increasing or enhancing baculovirus yield in a host cell, wherein said baculovirus does not normally encode a protein comprising the amino acid sequence set forth in SEQ ID NO:1 or a fragment or variant thereof, said method including the step of engineering said host cell to express one or more proteins comprising the amino acid sequences set forth in SEQ ID NOS:1, 3 or 4, or fragments or variants thereof, to thereby increase or enhance baculovirus yield in the host cell.

In an embodiment of this aspect, said host cell is engineered to express SEQ ID NO:1.

In a second aspect of the first broad form, the invention provides a method for increasing or enhancing baculovirus yield in a host cell, wherein said baculovirus does not normally encode a protein comprising the amino acid sequence set forth in SEQ ID NO:2 or a fragment or variant thereof, said method including the step of engineering said host cell to express a protein comprising the amino acid sequence set forth in SEQ ID NO:2 or a fragment or variant thereof, to thereby increase or enhance baculovirus yield in the host cell.

In a third aspect of the first broad form, the invention provides a method for increasing or enhancing baculovirus yield in a host cell, said method including the step of engineering said host cell to express a protein comprising the amino acid sequence set forth in SEQ ID NO:3 or 4, or a fragment or variant thereof, to thereby increase or enhance baculovirus yield in the host cell.

In certain embodiments, the abovementioned aspects may be suitable for increasing recombinant protein expression in a host cell.

Also provided is an isolated, baculovirus-infected host cell produced according to the method of the aforementioned aspects.

In a fourth aspect of the first broad form, the invention provides an isolated, baculovirus-infected host cell, wherein said host cell is engineered to express one or more of the proteins comprising the amino acid sequences set forth in SEQ ID NOS:1, 3 or 4, or fragments or variants thereof.

In an embodiment of this aspect, said baculovirus does not normally encode a protein comprising the amino acid sequence set forth in SEQ ID NO:1 or a fragment or variant thereof,

In a particular embodiment, said host cell is engineered to express SEQ ID NO:1.

In an alternative embodiment, said baculovirus normally encodes a protein comprising the amino acid sequence set forth in SEQ ID NO:1 or a fragment or variant thereof.

In a fifth aspect of the first broad form, the invention provides an isolated, baculovirus-infected host cell, wherein said baculovirus does not normally encode a protein comprising the amino acid sequence set forth in SEQ ID NO:2 or a fragment or variant thereof, and said host cell is engineered to express a protein comprising the amino acid sequence set forth in SEQ ID NO:2 or a fragment or variant thereof.

In an alternative aspect, said baculovirus normally encodes a protein comprising the amino acid sequence set forth in SEQ ID NO:2 or a fragment or variant thereof.

In a sixth aspect of the first broad form, the invention provides an isolated, baculovirus-infected host cell, wherein said host cell is engineered to express a protein comprising the amino acid sequences set forth in SEQ ID NOS:3 or 4, or a fragment or variant thereof.

In a further aspect, the invention provides a method for producing an isolated host cell suitable for infection by a baculovirus, said method including engineering a host cell such that said host cell expresses two or more of the proteins comprising the amino acid sequences set forth in SEQ ID NOS:1, 3 or 4, or fragments or variants thereof, to thereby produce the isolated host cell suitable for infection by a baculovirus.

In an embodiment, the host cell is capable of expressing a protein comprising the amino acid sequence set forth in SEQ ID NO:1 or a fragment or variant thereof, and is engineered to express one or more of proteins comprising the amino acid sequences set forth in SEQ ID NOS:3 or 4, respectively, or fragments or variants thereof.

In another further aspect, the invention provides a method for producing an isolated host cell suitable for infection by a baculovirus, said method including engineering a host cell such that said host cell expresses:

(i) at least one of the proteins comprising the amino acid sequences set forth in SEQ ID NOS:1, 3 or 4, or fragments or variants thereof; and

(ii) a protein comprising the amino acid sequence set forth in SEQ ID NO:2 or a fragment or variant thereof,

to thereby produce the isolated host cell suitable for infection by a baculovirus.

In an embodiment, the host cell is capable of expressing a protein comprising the amino acid sequence set forth in SEQ ID NO:1 or a fragment or variant thereof, and is engineered to express one or more proteins comprising the amino acid sequences set forth in SEQ ID NOS:2-4, respectively, or fragments or variants thereof.

In an embodiment, the host cell is capable of expressing a protein comprising the amino acid sequence set forth in SEQ ID NO:2 or a fragment or variant thereof, and is engineered to express one or more proteins comprising the amino acid sequences set forth in SEQ ID NOS:1, 3 or 4, or a fragments or variants thereof.

In certain embodiments of the preceding further aspects, said host cell may be infected by a baculovirus.

Also provided is an isolated host cell produced according to the method of the aforementioned aspects.

In yet another aspect, the invention provides an isolated host cell suitable for infection by a baculovirus, wherein said host cell is capable of expressing two or more of the proteins comprising SEQ ID NOS:1, 3 or 4, or fragments or variants thereof.

In still yet another aspect, the invention provides an isolated host cell suitable for infection by a baculovirus, wherein said host cell is capable of expressing:

(i) at least one of the proteins comprising the amino acid sequences set forth in SEQ ID NOS:1, 3or 4, or fragments or variants thereof, and

(ii) a protein comprising the amino acid sequence set forth in SEQ ID NO:2 or a fragment or variant thereof.

In certain embodiments of these aspects, said isolated host cell may be infected by a baculovirus.

Suitably, the isolated host cell is capable of increased or enhanced baculovirus yield. In some embodiments, the host cell may be capable of facilitating enhanced recombinant protein expression by a baculovirus.

In a yet further aspect, the invention provides a method for producing a baculovirus, said method including the step of cultivating a host cell of the aforementioned aspects that comprises a baculovirus, to thereby produce the baculovirus.

In a still yet further aspect, the invention provides a method for producing a recombinant protein from a recombinant baculovirus, said method including the step of cultivating a host cell of the aforementioned aspects that comprises a recombinant baculovirus, to thereby produce the recombinant protein from the baculovirus.

In certain embodiments of the first broad form, the method includes the step of infecting a host cell with a virus to thereby express said one or more proteins in the host cell, wherein the virus with which the host cell is infected to express said one or more proteins in the host cell is a different virus than the baculovirus for which yield is increased or enhanced in the host cell.

In preferred said embodiments, the virus with which the host cell is infected to express said one or more proteins in the host cell is a baculovirus.

In certain embodiments, an isolated host cell provided according to the first broad form has been infected by a virus to thereby express said one or more proteins in the host cell, and further infected by a baculovirus, wherein said baculovirus is not the virus with which the host cell is infected to express said one or more proteins in the host cell.

In a second broad form, the invention provides isolated proteins that may be useful for expression in host cells to increase or enhance baculovirus yield.

In a first aspect of the second broad form, the invention provides an isolated protein comprising the amino acid sequence set forth in SEQ ID NOS:3 or 4, or a fragment, variant, or derivative thereof. Preferably said protein consists of, or consists essentially of the amino acid sequence set forth in SEQ ID NOS:3 or 4, or a fragment, variant, or derivative thereof.

In a second aspect of the second broad form, the invention provides an isolated nucleic acid encoding the isolated protein, or fragment, variant or derivative thereof, of the first aspect. Preferably said nucleic acid comprises the nucleotide sequence set forth in SEQ ID NO:7 or SEQ ID NOS:8-9.

This aspect also includes fragments, variants, and derivatives of said isolated nucleic acid.

In a third aspect of the second broad form, the invention provides a genetic construct comprising an isolated nucleic acid of the third aspect.

In a fourth aspect of the second broad form, the invention provides an isolated host cell engineered to express one or more of the proteins of the first aspect, and/or a nucleic acid of the third aspect. In one preferred embodiment of this aspect, said host cell comprises a genetic construct of the third aspect.

In a fifth aspect of the second broad form, the invention provides an antibody or antibody fragment that binds or is raised against the isolated protein of the first aspect, wherein said antibody does not bind the amino acid sequence set forth in SEQ ID NO:1.

It will be appreciated that the indefinite articles “a” and “an” are not to be read as singular indefinite articles or as otherwise excluding more than one or more than a single subject to which the indefinite article refers. For example, “a” protein includes one protein, one or more proteins or a plurality of proteins.

As used herein, unless the context requires otherwise, the words “comprise”, “comprises” and “comprising” will be understood to mean the inclusion of a stated integer or group of integers but not the exclusion of any other integer or group of integers.

### BRIEF DESCRIPTION OF THE FIGURES

In order that the invention may be readily understood and put into practical effect, preferred embodiments will now be described by way of example with reference to the accompanying figures, wherein:

FIG. 1 sets out SEQ ID NOS:1-9.

FIG. 2 sets out the p35-trunc-tail protein and encoding gene, and changes relative to the wild type p35 protein and encoding gene from AcMNPV.

FIG. 3 sets out a schematic view of the pIZ/V5-His vector used for production of HzAM1 cells stably expressing p35-trunc-tail. Note that the same OpIE2 promoter is used to express the gene of interest (for which there are several insertion sites included), and the Zeocin™ resistance gene (Invitrogen life technologies, 2010).

FIG. 4 sets out Zeocin kill curves of three individual lots of stock (non-transfected) HzAM1 cells.

FIG. 5 sets out the structure of Zeocin (phleomycin D1).

FIG. 6 sets out total cell density for non-infected (i) HzAM1 and (ii) stably transfected p35-trunc-tail HzAM1 cultures over a period corresponding to the HearNPV infection period, with ±15% error bars. This figure shows that the cells used for the infections set forth in FIG. 7 displayed normal exponential growth. The p35-trunc-tail transfected cells in this case had been weaned off Zeocin in preparation for the infection experiment.

FIG. 7 sets out mean total cell density for HearNPV infected, stably transfected p35-trunc-tail HzAM1, and non-transfected HzAM1, over the infection period, with ±15% error bars.

FIG. 8 sets out normalized polyhedrin mRNA expression levels for HearNPV infected, stably transfected p35-trunc-tail HzAM1 versus HearNPV infected non-transfected HzAM1.

FIG. 9 sets out normalized p35-trunc-tail mRNA expression levels for HearNPV infected, stably transfected p35-trunc-tail HzAM1 (0-3 dpi).

FIG. 10 sets out Western blot analysis of GFP expression in SD cells transfected with the reporter plasmid encoding GFP with or without dsRNA targeting GFP (ds), and subsequently mock-infected (-BV) or infected with AcMNPV (+BV) at 4, 8 and 24 hpi. Specific antibodies to GFP were used as probe and hsp70 antibody to show equal loading of samples.

FIG. 11 sets out: (A) Schematic diagram showing p35 deletion mutant constructs produced as described in the methods. (B) Western blot analysis of SD cells co-transfected with pIZ/p35 or mutant constructs and dsGFP using the anti-GFP antibody and anti-Hsp70 as control. (C) RT-PCR analysis of RNA from Sf9 cells infected with wild type AcMNPV or mutant Ap35-AcMNPV at 8 hpi, or transfected with pIZ empty vector, pIZ/p35 or either of the mutant constructs (as in A). Actin gene was used as control to show integrity of RNA. Specific primers to the p35 middle region (Table 4) were used in the PCRs. (D) Western blot analysis of Sf9 cells transfected with pIZ/GFP only, pIZ/GFP and dsGFP plus pIZ/p35 or pIZ/p35-V71P. The blot was first probed with the anti-GFP antibody and subsequently with the anti-Hsp70 antibody as control.

FIG. 12 sets out: (A) Western blot analysis of Sf9 cells transfected with pIZ/p35 or empty pIZ vector, then co-transfected with pIZ/GFP and dsGFP. (B) Western blot analysis of Aag2 cells transfected with pIZ/p35 or empty pIZ vector, then transfected with dsProhibitin. In (A) and (B), specific antibodies to GFP and prohibitin were used as probes, respectively, and hsp70 antibody to show equal loading of samples. (C) RT-qPCR analysis of RNA from Aag2 cells treated as in (B) using specific primers to prohibitin (Table 4) (D) RT-qPCR analysis of RNA from Sf9, S2 and HzFB cells treated as in (A) using specific primers to the GFP gene (Table 4).

FIG. 13 sets out: (A) RT-qPCR analysis of RNA from Vero cells transfected with pEGFP-N1 and dsEGFP or dsGATA4 (as control) in the absence or presence of pEGFPN1/p35 (p35) using specific primers to EGFP. (B) RT-qPCR analysis of RNA from NIH-3T3 cells transfected with pEGFP-N1/p35 with or without dsProhibitin using specific primers to prohibitin. There are statistically significant differences between groups with different letters at p<0.0001 in (A) and at p<0.05 in (B).

FIG. 14 sets out: Western blot analysis of Sf9 cells co-transfected with pIZ/GFP and dsGFP, then infected with mutant AcMNPV lacking the p35 gene (AP35) at various times after infection using anti-GFP antibody as probe. Control cells were infected with the wild-type (wt) AcMNPV and analysed at 24 hpi. Hsp70 antibody was used to show equal loading of samples and anti-gp64 antibody was used to monitor budded virus production.

FIG. 15 sets out: (A) Northern blot analysis of dsGFP levels at times post AcMNPV infection using a specific probe to GFP. rRNA is shown as loading control. (B) Northern blot analysis of dsGFP in Sf9 cells transfected with the pIZ empty vector and pIZ/p35. (C) Western blot analysis of Sf9 cells transfected with pIZ/GFP only or transfected with pIZ/GFP and GFP siRNAs (siGFP) plus mock, pIZ empty vector or pIZ/p35. The blot was probed with an anti-GFP antibody and anti-Hsp70 to show equal loading of the samples.

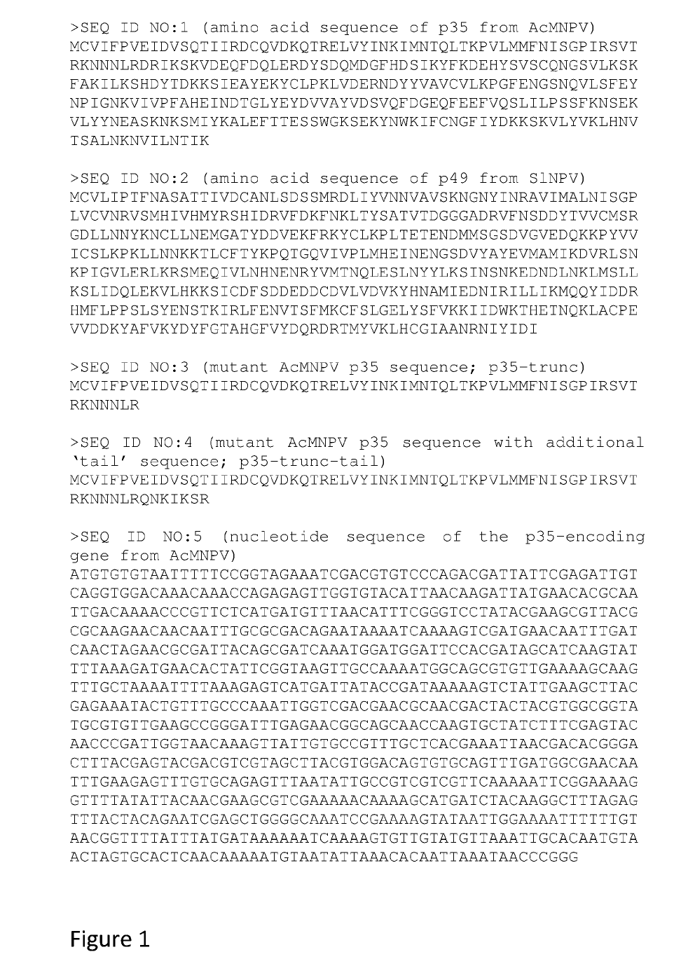

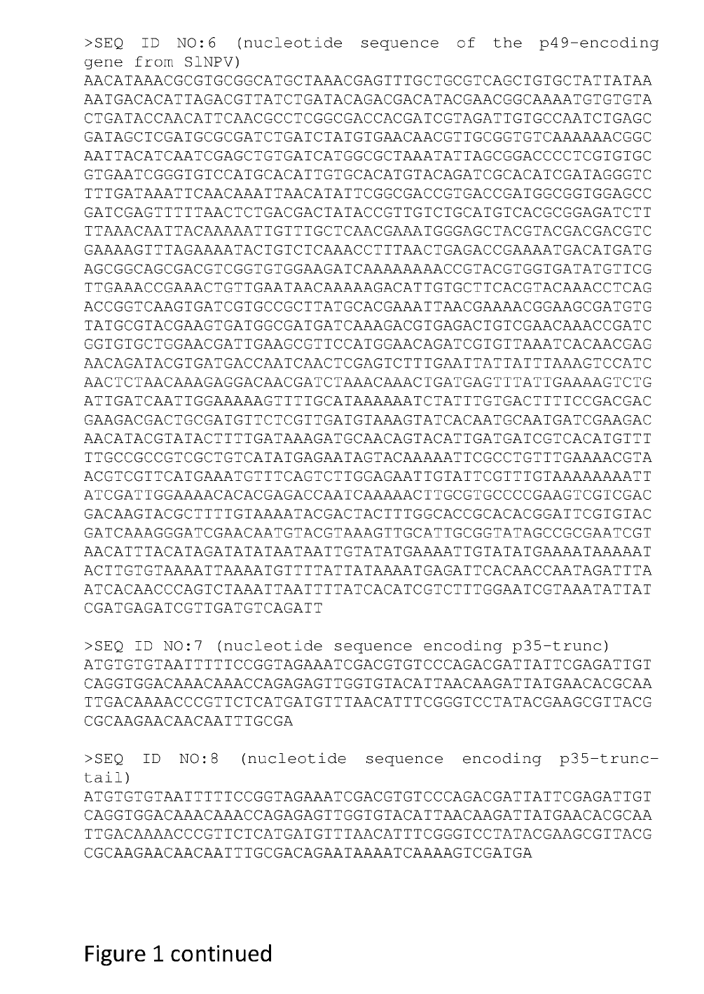

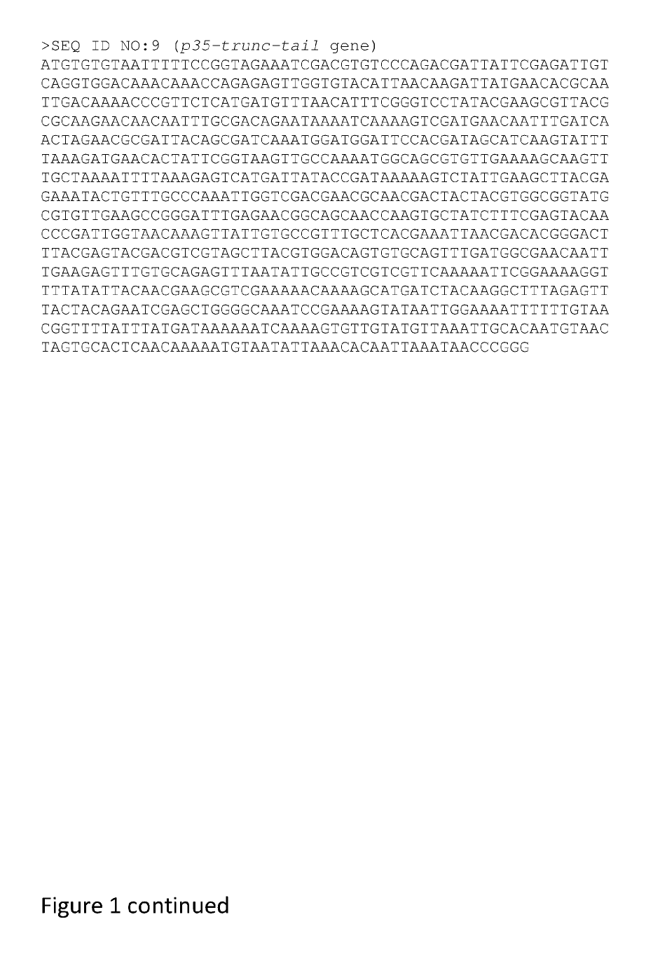

### BRIEF DESCRIPTION OF THE SEQUENCE LISTING

SEQ ID NO:1 Amino acid sequence of the p35 protein from AcMNPV.
SEQ ID NO:2 Amino acid sequence of the p49 protein from S1NPV.
SEQ ID NO:3 Amino acid sequence of the mutant p35 protein ‘p35-trunc’.
SEQ ID NO:4 Amino acid sequence of the mutant p35 protein ‘p35-trunc-tail’
SEQ ID NO:5 Nucleotide sequence of the p35 gene from AcMNPV.
SEQ ID NO:6 Nucleotide sequence of the p49 gene from SlNPV.
SEQ ID NO:7 Nucleotide sequence of a p35-trunc -encoding Open Reading Frame.
SEQ ID NO:8 Nucleotide sequence of a p35-trunc-tail-encoding Open Reading Frame.
SEQ ID NO:9 Nucleotide sequence of the p35-trunc-tail gene.

### DETAILED DESCRIPTION

The invention is at least partly predicted on the surprising discovery that the expression of a p35 protein in baculovirus-infected host cells may lead to an increase in baculovirus yield, wherein said baculovirus does not normally encode a p35 protein.

The invention is also at least partly predicted on the surprising discovery of certain mutant p35 proteins that may be useful for expression in host cells to increase baculovirus yield from said host cells.

The invention therefore broadly provides means for increasing baculovirus yield and/or the expression of a recombinant protein from a baculovirus, in host cells.

### Isolated Proteins and Uses Thereof

For the purposes of this invention, by “isolated” is meant material (e.g. proteins, nucleic acids, cells etc) that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

By “protein” is meant an amino acid polymer, comprising natural and/or non-natural amino acids, including L- and D-isomeric forms as are well understood in the art.

p35, p49, p35-trunc, and p35-trunc-Tail

Certain aspects of the invention relate to engineering host cells to express an isolated protein comprising the amino acid sequence set forth in SEQ ID NO:1, henceforth referred to as a “p35” protein, or fragments, variants, or derivatives thereof.

Other aspects of the invention relate to engineering host cells to express an isolated protein comprising the amino acid set forth in SEQ ID NO:2, henceforth referred to as a “p49” protein, or fragments, variants or derivatives thereof. Without being bound by theory, it is speculated that p35 and p49 may be functionally related, having about 50% amino acid sequence identity.

Some aspects of the invention are directed to isolated proteins comprising the amino acid sequence set forth in SEQ ID NO:3 or SEQ ID NO:4, or fragments, variants, or derivates thereof. Related aspects are directed to engineering host cells to express said isolated proteins.

It will appreciated that the isolated protein comprising the amino acid sequence set forth in SEQ ID NO:3 is a particular fragment of the isolated protein comprising the amino acid sequence set forth in SEQ ID NO:1. Specifically, the amino acid sequence set forth in SEQ ID NO:3 consists of the contiguous amino acid residues 1-64 of SEQ ID NO:1. As used herein, the particular p35 fragment set forth in SEQ ID NO:3 will be referred to as a “p35-trunc” protein.

It will be further appreciated that the isolated protein comprising the amino acid sequence set forth in SEQ ID NO:4 is a particular variant of the isolated protein comprising the amino acid sequence set forth in SEQ ID NO:1. Specifically, the amino acid sequence set forth consists of (i) the contiguous amino acid residues 1-64 of SEQ ID NO:1; and (ii) the amino acid residues QNKIKSR at positions 65-71. As used herein, the particular amino acid sequence set forth in SEQ ID NO:4 will be referred to as a “p35-trunc-tail” protein.

Without being bound by theory, it is hypothesized that p35, p35-trunc, and/or p35-trunc-tail may exhibit at least some shared biological activity. In this respect, it is expected that at least some of the functional properties of p35, particularly those of or enabled by the N-terminal region of the protein, may be conserved or substantially conserved, among p35, p35-trunc, and p35-trunc-tail.

It is further hypothesized that p35, p35-trunc, and/or p35-trunc-tail may exhibit at least some differences in biological activity. In this respect, it is expected that at least some of the functional properties of p35, particularly those of or enabled by regions of the protein outside of the N-terminal region, may be absent, or substantially absent, in p35-trunc and p35-trunc-tail. Furthermore, it is expected that at least some of the functional properties, if any, of p35-trunc-tail that are of or enabled by the presence of variant amino acids at positions 65-71 may be absent, or substantially absent, in p35 and p35-trunc.

Other aspects of the invention relate to engineering host cells to express more than one of the aforementioned proteins, or fragments, variants or derivatives thereof.

As herein described, certain embodiments of the invention relate to isolated fragments of p35, p35-trunc, p35-trunc-tail, or p49 proteins.

In some embodiments, a protein “fragment” includes an amino acid sequence which constitutes less than 100%, but at least 20%, preferably at least 30%, more preferably at least 80% or even more preferably at least 90%, 95%, 96%, 97%, 98% or 99% of the amino acid sequence set forth in SEQ ID NOS:1-4, respectively.

In some embodiments, a protein fragment comprises no more than 10, 12, 15, 20, 25, 30, 35, 40, 45, 50, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, or 285 contiguous amino acids of SEQ ID NO:1.

As described above, the p35-trunc amino acid sequence set forth in SEQ ID NO:3 is also one particular fragment of p35, consisting of 64 contiguous amino acids of the p35 sequence set forth in SEQ ID NO:1.

In some embodiments, a protein fragment comprises no more than 10, 12, 15, 20, 25, 30, 35, 40, 45, 50, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440 or 445 contiguous amino acids of SEQ ID NO:2.

In some embodiments, a protein fragment comprises no more than 10, 11, 12, 13, 14, 15, 16, 17, 18, 19, 20, 21,22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 contiguous amino acids of SEQ ID NO:3

In some embodiments, a protein fragment comprises no more than 10, 11, 12, 13, 14, 15, 16, 17, 18, 19, 20, 21,22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 contiguous amino acids of SEQ ID NO:4

As hereinabove described, certain embodiments of the invention relate to isolated variants of the p35, p35-trunc, p35-trunc-tail, or p49 protein. It will be appreciated that p35, p35-trunc, p35-trunc-tail, or p49 protein variants according to the invention may also be protein fragments.

As used herein “variant” proteins of the invention have one or more amino acids deleted or substituted by different amino acids. It is well understood in the art that some amino acids may be substituted or deleted without changing the activity of the protein (“conservative” substitutions). More substantial changes to activity may be made by introducing substitutions or deletions that are less conservative (“non-conservative” substitutions). Variants include naturally occurring (e.g., allelic) variants, orthologs (i.e. from other viruses) and synthetic variants, such as produced in vitro using mutagenesis techniques.

In certain preferred embodiments, protein variants share at least 70% or 75%, preferably at least 80% or 85% or more preferably at least 90%, 91%, 92%, 93%, 94%, 95%, 96%, 97%, 98% or 99% sequence identity with an amino acid sequence of the isolated protein comprising the amino acid sequence set forth in SEQ ID NOS:1-4, respectively.

As hereinabove described, it will be appreciated that the p35-trunc-tail amino acid sequence set forth in SEQ ID NO:4 is also one particular p35 variant, consisting of 64 contiguous amino acids of the p35 sequence set forth in SEQ ID NO:1, and variant amino acid residues at positions 65-71.

Terms used generally herein to describe sequence relationships between respective proteins and nucleic acids include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

In some embodiments, a p35, p35-trunc, p35-trunc-tail, or p49 protein fragment or protein variant, as described above, may be a “biologically active” fragment or variant, which retains biological activity of said protein.

The biologically active fragment of a p35, p35-trunc, p35-trunc-tail, or p49 protein described herein preferably has greater than 10%, preferably greater than 20%, more preferably greater than 50% and even more preferably greater than 75%, 80%, 85%, 90% 95%, 96%, 97%, 98% or 99% of a biological activity of the protein comprising the amino acid sequence set forth in SEQ ID NO:1, SEQ ID NO:3, SEQ ID NO:4, or SEQ ID NO:2, respectively.

One non-limiting example of biological activity of a p35, p35-trunc, p35-trunc-tail, and/or p49 protein may be inhibition of apoptosis of a host cell (including inhibition of caspase activity of a host cell, although without limitation thereto).

Another non-limiting example of biological activity of a p35, p35-trunc, p35-trunc-tail, and/or p49 protein may be “viral suppressor of RNAi” or “VSR” activity. As will be understood by the skilled person, “viral suppressor of RNAi” activity refers to an ability of a viral protein to suppress or inhibit host cell RNAi pathway(s).

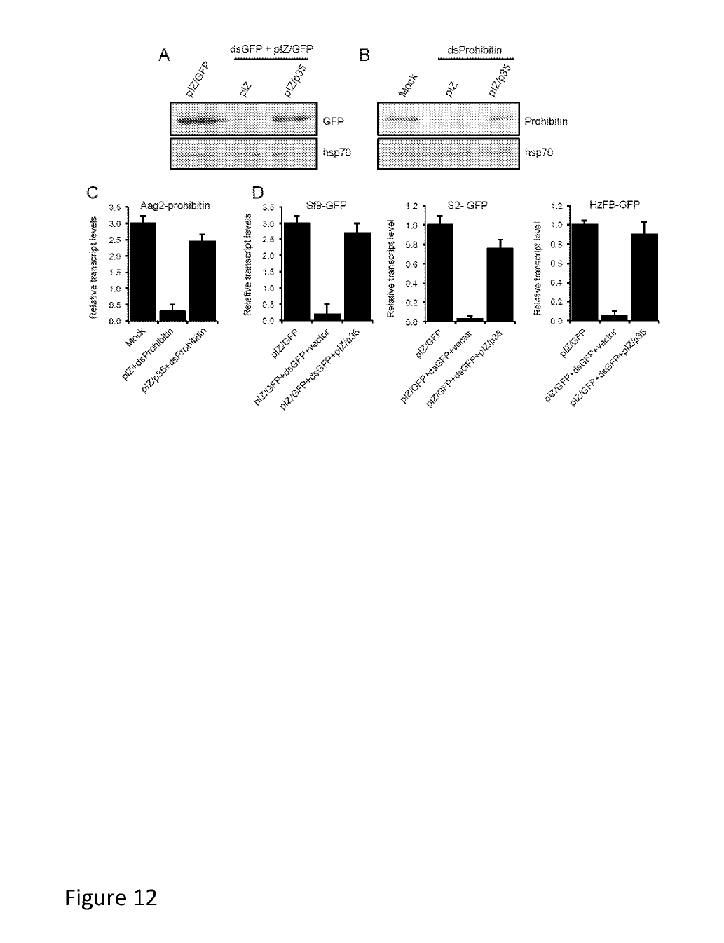

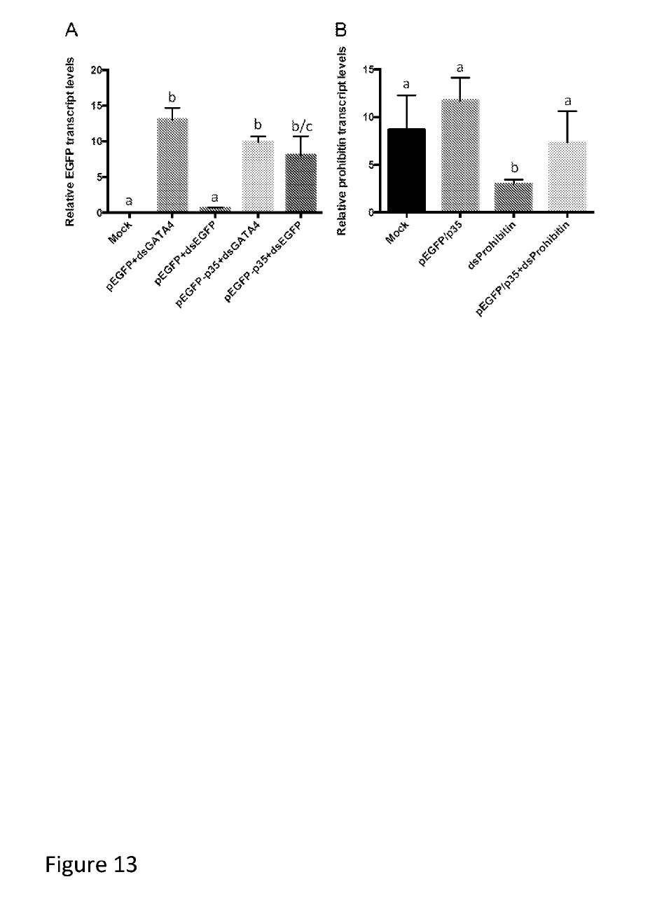

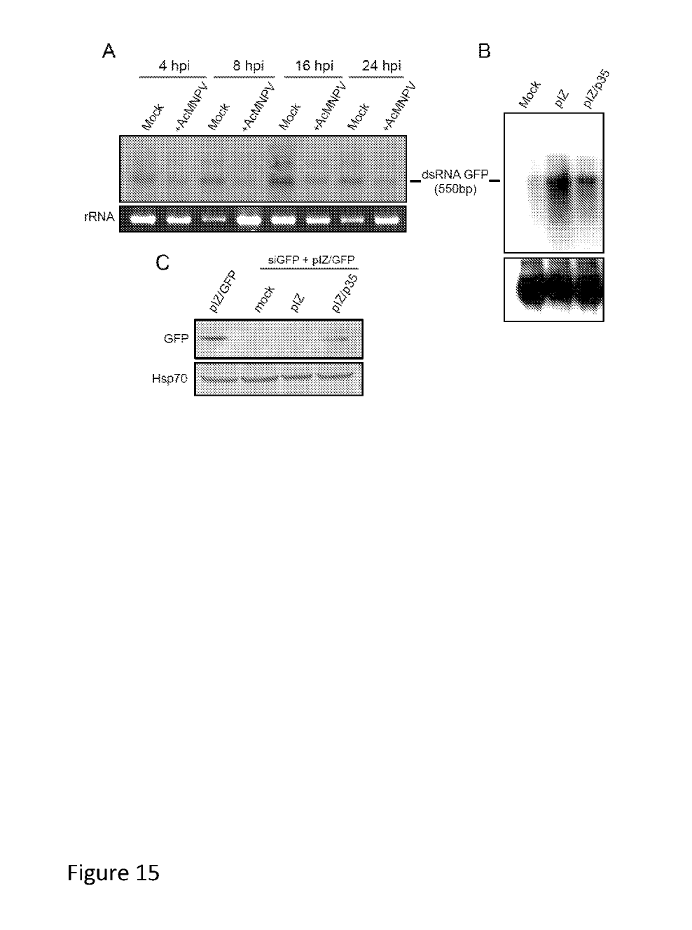

In some embodiments, VSR activity of one or more of the aforementioned proteins may occur downstream of Dicer-2 processing by the host cell machinery. As will be evident from the Examples and FIGS. 12-13, p35 can inhibit host cell RNAi activity in host cells. It will also be evident from the Examples and FIG. 15 that the VSR effect of p35 may occur after processing of dsRNA into siRNA by Dicer-2.

In regard to biological activity of the protein fragments and protein variants described herein, as hereinbefore described, it is expected that at least some functional properties may be conserved among p35, p35-trunc, and p35-trunc-tail. Furthermore, p35 and p49 may be functionally related, having about 50% amino acid sequence identity.

It is further speculated that p35-trunc may possess one or more functional differences as compared to p35. In certain embodiments, a p35 fragment may comprise substantially the same functional properties as p35-trunc.

Similarly, it is speculated that p35-trunc-tail may possess one or more functional differences as compared to p35 and/or p35-trunc. In certain embodiments, a p35 variant may comprise substantially the same functional properties as p35-trunc-tail.

Certain embodiments of the invention may relate to derivatives of an isolated protein described herein. As used herein, “derivative” proteins are proteins of the invention that have been altered, for example by conjugation or complexing with other chemical moieties or by post-translational modification techniques as would be understood in the art. Such derivatives include amino acid deletions and/or additions to polypeptides of the invention, or variants thereof.

“Additions” of amino acids may include fusion of the peptide or polypeptides of a p35, p35-trunc, p35-trunc-tail, or p49 protein, or variants thereof, with other peptides or polypeptides. Particular examples of such peptides include amino (N) and carboxyl (C) terminal amino acids added for use as fusion partners or “tags”.

Well-known examples of fusion partners include hexahistidine (6X-HIS)-tag, N-Flag, Fc portion of human IgG, glutathione-S-transferase (GST) and maltose binding protein (MBP), which are particularly useful for isolation of the fusion polypeptide by affinity chromatography. For the purposes of fusion polypeptide purification by affinity chromatography, relevant matrices for affinity chromatography may include nickel-conjugated or cobalt-conjugated resins, fusion polypeptide specific antibodies, glutathione-conjugated resins, and amylose-conjugated resins respectively. Some matrices are available in “kit” form, such as the ProBond™ Purification System (Invitrogene Corp.) which incorporates a 6X-His fusion vector and purification using ProBond™ resin.

The fusion partners may also have protease cleavage sites, for example enterokinase (available from Invitrogen Corp. as EnterokinaseMax™), Factor Xₐ or Thrombin, which allow the relevant protease to digest the fusion polypeptide of the invention and thereby liberate the recombinant polypeptide of the invention therefrom. The liberated polypeptide can then be isolated from the fusion partner by subsequent chromatographic separation.

Fusion partners may also include within their scope “epitope tags”, which are usually short peptide sequences for which a specific antibody is available.

Other derivatives that may be used for the invention include those incorporating unnatural amino acids and/or their derivatives during peptide or polypeptide synthesis. Non-limiting examples of incorporating unnatural amino acids and derivatives during peptide synthesis include, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

### Isolated Nucleic Acids and Use Thereof

The term “nucleic acid” as used herein designates single-or double-stranded DNA and RNA. DNA includes genomic DNA and cDNA. RNA includes mRNA, RNA, miRNA, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylycytosine, methylinosine, methyladenosine and/or thiouridine, although without limitation thereto.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

Some aspects of the invention provide isolated nucleic acids encoding an isolated p35-trunc or p35-trunc-tail protein, or fragment, variant or derivative thereof Preferably said nucleic acid comprises the nucleotide sequence set forth in SEQ ID NO:7 or SEQ ID NOS:8-9.

Aspects of the invention also relate to engineering host cells to express isolated nucleic acids encoding isolated p35, p35-trunc, p35-trunc-tail, and/or p49 proteins as described herein, inclusive of fragments, variants and derivatives of the isolated protein.

A nucleic acid encoding a p35 protein is exemplified in SEQ ID NO:5. A nucleic acid encoding a p49 protein is exemplified in SEQ ID NO:6.

In some embodiments, these aspects relate to isolated fragments of a nucleic acid that encodes an isolated p35, p35-trunc, p35-trunc-tail, or p49 protein, variant, fragment of derivative described herein. In preferred embodiments, nucleic acid fragments include an nucleic acid sequence which constitutes less than 100%, but at least 20%, preferably at least 30%, more preferably at least 80% or even more preferably at least 90%, 95%, 96%, 97%, 98% or 99% of the nucleotide sequence set forth in SEQ ID NOS:5-9, respectively.

In some embodiments, these aspects relate to isolated variants of a nucleic acid that encodes an isolated p35, p35-trunc, p35-trunc-tail, or p49 protein variant, fragment or derivative described herein. In preferred embodiments, nucleic acid variants share at least 60% or 65%, preferably at least 70% or 75%, more preferably at least 80%, 85%, 90%, or at least 91%, 92%, 93%, 94%, 95%, 96%, 97%, 98%, or 99% nucleotide sequence identity with an isolated nucleic acid that encodes SEQ ID NOS:1-4, respectively.

In yet another embodiment, nucleic acid variants hybridize to an isolated nucleic acid that encodes SEQ ID NOS:1-4, respectively, under at least low stringency conditions, preferably under at least medium stringency conditions and more preferably under high stringency conditions.

“Hybridize and Hybridization” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing between complementary purines and pyrimidines as are well known in the art.

In this regard, it will be appreciated that modified purines (for example, inosine, methylinosine and methyladenosine) and modified pyrimidines (thiouridine and methylcytosine) may also engage in base pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“High stringency conditions” designates those conditions under which only nucleic acid having a high frequency of complementary bases will hybridize.
Reference herein to high stringency conditions include and encompass:
(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;
(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and
(iii) 0.2×SSC, 0.1% SDS for washing at or above 68° C. for about 20 minutes.

In general, washing is carried out at Tₘ=69.3+0.41 (G+C) % −12° C. In general, the Tₘ of a duplex DNA decreases by about 1° C. with every increase of 1% in the number of mismatched bases.

Notwithstanding the above, stringent conditions are well known in the art, such as described in Chapters 2.9 and 2.10 of. Ausubel et al., supra. A skilled addressee will also recognize that various factors can be manipulated to optimize the specificity of the hybridization. Optimization of the stringency of the final washes can serve to ensure a high degree of hybridization.

In other embodiments, isolated nucleic acid variants may be produced using a nucleic acid amplification technique. Suitable nucleic acid amplification techniques are well known to the skilled addressee, and include polymerase chain reaction (PCR); strand displacement amplification (SDA); rolling circle replication (RCR); nucleic acid sequence-based amplification (NASBA), Q-β replicase amplification and helicase-dependent amplification, although without limitation thereto.

As used herein, an “amplification product” refers to a nucleic acid product generated by nucleic acid amplification.

Particularly for analytical purposes, nucleic acid amplification techniques may include quantitative and semi-quantitative techniques such as qPCR, real-time PCR and competitive PCR, as are well known in the art.

Suitably, isolated nucleic acid variants may be produced using nucleic acid amplification techniques using one or more degenerate primers based on, or derived from, a nucleotide sequence of an isolated nucleic acid disclosed herein. By way of example, the degenerate primer(s) may be designed to anneal to one or more nucleotide sequences of a variant nucleic acid to thereby facilitate amplification of the variant nucleic acid, or a fragment thereof.

### Genetic Constructs and Transfection of Host Cells

Certain aspects of the invention relate to genetic constructs that comprise one or more isolated nucleic acids encoding one or more of:

(i) a p35 protein;

(ii) a p35-trunc protein;

(iii) a p35-trunc-tail protein; and

(iv) a p49 protein,

and one or more additional nucleotide sequences.

Suitably, the genetic construct may be in the form of, or comprise genetic components of, a plasmid, bacteriophage, a cosmid, a yeast or bacterial artificial chromosome as are well understood in the art.

Genetic constructs may be suitable for maintenance and propagation of an isolated nucleic acid in bacteria or other host cells, for manipulation by recombinant DNA technology and/or expression of the nucleic acid or an encoded protein as herein described.

For the purposes of host cell expression, the genetic construct may be an expression construct. Suitably, the expression construct comprises one or more nucleic acids or variants described herein operably linked to one or more additional sequences in an expression vector.

An “expression vector” may be either a self-replicating extra-chromosomal vector such as a plasmid, or a vector that integrates into a host genome.

By “operably linked” is meant that said additional nucleotide sequence(s) is/are positioned relative to the nucleic acid of the invention preferably to initiate, regulate or otherwise control transcription.

In one embodiment, the additional nucleotide sequences are regulatory sequences. Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences.

Constitutive or inducible promoters as known in the art may be used for genetic constructs of the invention. The promoters may be either naturally occurring promoters, or hybrid promoters that combine elements of more than one promoter.

In certain other preferred embodiments, a genetic construct of the invention is a genetic construct for “genome editing”. As will be appreciated by the skilled person, “genome editing” is a method for mutagenesis or genetic modification in which DNA is inserted, substituted, modified, or deleted from the genetic material of an organism in a targeted manner, using engineered nucleases.

Methods for genome editing include “zinc finger nuclease” methods, as described for example by Miller et al., 2007, Nat. Biotech. 25 778; “CRISPR/Cas” methods, as described for example by Cong et al., Science 339 819; and “TALEN” methods, as described for example by Bedell et al., Nature 491, 114.

As will be understood by those skilled in the art, genome editing of a host cell includes the transformation of a cell with one or more genetic constructs facilitating the expression of:

(i) one or more DNA nucleases; and

(ii) one or more molecules that guide the cleavage of DNA at a targeted region within the genetic material of an organism by said nuclease(s).

Targeted DNA breaks are thereby induced in the genetic material of the organism. Said targeted DNA breaks are generally double stranded DNA breaks, although without limitation thereto.

In embodiments of genome editing wherein a zinc finger nuclease method is used, the one or more molecules that guide the cleavage of DNA at a targeted region within the genetic material of an organism by said nuclease(s) are proteins comprising a zinc finger DNA-binding domain. Generally, a plurality of said proteins are fused to said nuclease(s), and the plurality of zinc finger DNA-binding domains of said proteins bind with at least partial specificity to the targeted region, and thereby induce cleavage of the targeted region by said nuclease(s).

In embodiments of genome editing wherein a TALEN method is used, the one or more molecules that guide the cleavage of DNA at a targeted region within the genetic material of an organism by said nuclease(s) are proteins comprising a transcription activator-like effector DNA-binding (“TALE”) domain. Generally, a plurality of said proteins are fused to said nuclease(s), and the plurality of TALE DNA-binding domains of said proteins bind with at least partial specificity to the targeted region, and thereby induce cleavage of the targeted region by said nuclease(s).

In embodiments of genome editing wherein a CRISPR/Cas method is used, the nuclease is a CRISPR-associated (Cas) nuclease, and the one or more molecules that guide the cleavage of DNA at a targeted region is a “guide” RNA molecule (or “gRNA”) with homology to the targeted region. Generally, the gRNA molecule forms a complex with the Cas nuclease and guides binding of the Cas nuclease to the targeted region with at least partial specificity, and thereby induces cleavage of the targeted region by said Cas nuclease.

It will be further understood that targeted DNA breaks induced by genome editing can facilitate non-homologous end joining or homology-dependent repair.

“Non-homologous end joining” is a cellular mechanism for DNA break repair wherein cleaved DNA ends are ligated, which is typically “error prone”, i.e introduces nucleotide sequence variation, e.g. insertions or deletions, at the site of the DNA break. DNA breakage followed by error-prone non-homologous end joining induced by genome editing can be used to inactivate targeted regions within the genetic material of organisms including plants and animals (as described for example by Gaj et al., 2013 Trends Microbiol. 31 397).

“Homology-dependent repair” is a cellular mechanism for DNA break repair wherein a nucleic acid possessing homology to the region surrounding a DNA break is used as a template for repair of the DNA break. Genome editing can be used to introduce nucleic acid variants into targeted regions within the genetic material of organisms including plants and animals (as described for example by Gaj et al., 2013 Trends Microbiol 31 397) by inducing DNA breakage followed by homology-dependent repair in the presence of a “donor molecule”, wherein said donor molecule comprises homology to the region surrounding the DNA break.

Suitably, according to these aspects, genome editing may be used to introduce a nucleic acid encoding one or more of a p35, p35-trunc, p35-trunc-tail, and a p49 protein of the invention, such as hereinbefore described, into the genetic material of a host cell, to thereby express said protein in said host cell. In certain preferred embodiments, a genetic construct of the invention is suitable for expression of an isolated nucleic acid described herein in an arthropod host cell.

In certain preferred embodiments, a genetic construct of the invention is suitable for expression of an isolated nucleic acid described herein in an insect host cell.

In preferred embodiments wherein an genetic construct of the invention is for expression of an isolated nucleic acid encoding a p35, p35-trunc, p35-trunc-tail, and/or p49 protein in an insect host cell, said insect cell is selected from the following group: Spodoptera frugiperda (“Sf”) and cell clones and/or cell populations derived from these cells, e.g. Sf9 and Sf21 cells; Helicoverpa zea (“Hzea”) and cell clones and/or cell populations derived from these cells, e.g. HzAM1; Trichoplusia ni (“Tni”) and cell clones and/or cell populations derived from these cells, e.g. Hi5 and TN368; Plutella xylostella (“Px”) and cell clones and/or cell populations derived from these cells, Anticarsia gemmatalis (“Ag”) and cell clones and/or cell populations derived from these cells, e.g. saUFL-AG-286 and UFL-AG-286; and Heteronychus arator and cell clones and/or cell populations derived from these cells, e.g. DSIR-HA-1179. For further non-limiting information on suitable insect cell lines for the production of baculoviruses, the skilled person is directed to van Oers and Lynn, 2010 Encyclopedia of Life Sciences (ELS). John Wiley & Sons, Ltd., Chichester.

In certain embodiments a genetic construct as herein described may comprise a selectable marker gene to allow the selection of transformed or transfected host cells. Selectable marker genes are well known in the art and will vary with the host cell used. As will be understood by one skilled in the art, certain selectable markers are particularly useful for the purpose of selection of transformed insect cells (such as neomycin resistance, hygromycin resistance, G418 resistance, zeocin and puromycin resistance).

The genetic construct may also include an additional nucleotide sequence encoding a fusion partner (typically provided by the expression vector) so that a recombinant polypeptide as described herein is expressed as a fusion protein, as hereinbefore described.

Introduction of genetic constructs for the expression of p35, p35-trunc, p35-trunc-tail, and/or p49 proteins into suitable host cells may be by way of techniques including but not limited to electroporation, heat shock, calcium phosphate precipitation, DEAE dextran-mediated transfection, liposome-based transfection (e.g. lipofectin, lipofectamine, Cellfectin), microinjection or microparticle bombardment, as are well known in the art.

As will be understood by those skilled in the art, transformation or transfection of a host cell with a genetic construct may be either “transient” or “stable”.

In general, “stable” transfection comprises the incorporation of a genetic construct into the genetic material of a host cell, wherein the genetic material comprising the genetic construct can be inherited to the progeny of said cell.

In general, “transient” transfection comprises the introduction of a genetic construct into a host cell without the incorporation of said genetic construct into the genetic material of said cell. Suitably, the genetic construct may be expressed using the cellular machinery within the host cell.

Increased Baculovirus Yields and/or Recombinant Protein Expression

Certain aspects of the invention are directed to increasing or enhancing baculovirus yield and/or recombinant protein expression in a host cell, by engineering said host cell to express a p35, p35-trunc, and/or p35-trunc-tail protein, to thereby increase or enhance baculovirus yield and/or recombinant protein expression in the host cell.

One aspect of the invention provides a method for increasing or enhancing baculovirus yield in a host cell, wherein said baculovirus does not normally encode a p35 protein, said method including the step of engineering the host cell to express a p35, p35-trunc, and/or p35-trunc-tail protein, to thereby increase or enhance baculovirus yield in the host cell.

As will be evident from the Examples, the yield of a Helicoverpa armigera nucleopolyhedrovirus (“HearNPV”) baculovirus that does not normally encode a p35 protein was substantially increased in a host cell engineered to express a p35-trunc-tail or p35 protein. The skilled person will therefore appreciate that the expression of a p35-trunc-tail protein, p35 protein, and/or a p35-trunc protein in a host cell may be particularly effective for increasing or enhancing the yield of a baculovirus in the host cell wherein the baculovirus does not normally encode a p35 protein. In this regard, as set forth above, it is hypothesized by the inventors that p35, p35-trunc, and p35-trunc-tail proteins may share at least some functional properties.

Without wishing to be bound by any particular theory, the inventors postulate that the response of a host cell to infection by a baculovirus that normally encodes a p35 protein (e.g. AcMNPV, although without limitation thereto) may be adapted to reduce or mitigate a biological effect of a p35, p35-trunc-tail, and/or p35-trunc protein on host cell defence pathways (e.g. on host cell production of antiviral RNAi, although without limitation thereto). In contrast, the response of a host cell to infection by a baculovirus that does not normally encode a p35 protein (e.g. HearNPV, although without limitation thereto) may not be adapted to reduce or mitigate said biological effect of a p35, p35-trunc-tail, and/or p35-trunc proteins on host cell defence pathways. Thus, the inventors postulate that host cell defences against a baculovirus that does not normally encode a p35 protein may be relatively ineffective for inhibiting or limiting the accumulation of the baculovirus in a host cell expressing a p35, p35-trunc-tail, or p35-trunc protein, which may lead to an increased or enhanced yield of said baculovirus in the host cell.

Another aspect of the invention provides a method for increasing or enhancing baculovirus yield in a host cell, said method including the step of engineering said host cell to express a p35-trunc or p35-trunc-tail protein, or a fragment or variant thereof, to thereby increase or enhance baculovirus yield in the host cell.

Yet another aspect of the invention provides a method for increasing or enhancing yield from a baculovirus by expressing p49 in a host cell.

One particular aspect provides a method for increasing or enhancing baculovirus yield in a host cell, wherein said baculovirus does not normally encode a p49 protein, said method including the step of engineering said host cell to express a p49 protein, to thereby increase or enhance the yield of the baculovirus in the host cell.

Without wishing to be bound by any particular theory, the inventors postulate that the response of a host cell to infection by a baculovirus that normally encodes a p49 protein (e.g. Spodoptera littoralis nucleopolyhedrovirus (“SlNPV”) although without limitation thereto) may be adapted to reduce or mitigate a biological effect of a p49 protein on host cell defence pathways (e.g. on host cell production of antiviral RNAi, although without limitation thereto). In contrast, the response of a host cell to infection by a baculovirus that does not normally encode a p49 protein (e.g. HearNPV, although without limitation thereto) may not be adapted to reduce or mitigate said biological effect of a p49 protein on host cell defence pathways. Thus, the inventors postulate that host cell defences against a baculovirus that does not normally encode a p49 protein may be relatively ineffective for inhibiting or limiting the accumulation of the baculovirus in a host cell expressing a p49 protein, which may lead to an increased or enhanced yield of said baculovirus in the host cell.

As used throughout this specification in the context of p35, p35-trunc, p35-trunc-tail, and p49 proteins, the term “normally encode” will be understood to mean that the wild type baculovirus encodes the protein. In contrast, the term “does not normally encode” will be understood to mean that the wild type baculovirus does not encode the protein.

As will be understood by one skilled in the art, a nucleotide sequence encoding a protein may be added, removed, and/or modified within the genetic material of a wild type baculovirus using standard techniques for the modification of nucleic acids, as herein described. By way of non-limiting example, the skilled person is directed to Clem et al., 1993, J Virol. 67 3730, wherein the production of a p35-null AcMNPV mutant is described.

Therefore, as used herein in the context of p35, p35-trunc, p35-trunc-tail, and/or p49 proteins, baculoviruses that “normally encode” the protein will be understood to include a modified baculovirus derived from a wild type baculovirus that encodes the protein, wherein the modified baculovirus does not encode the protein.

Furthermore, a baculovirus that “does not normally encode” the protein will be understood to include a modified baculovirus derived from a wild type baculovirus that does not encode the protein, wherein the modified baculovirus encodes the protein.

Additionally, as used herein in the context of p35, p35-trunc, p35-trunc-tail, and/or p49 proteins, a host cell that is “capable of expressing” the protein includes a host cell that comprises genetic material encoding the protein. Certain non-limiting examples of a host cell that is “capable of expressing” a p35, p35-trunc, p35-trunc-tail, and/or p49 protein include a host cell that has been transfected to express the protein (e.g. using a genetic construct as hereinbefore described), and a host cell that has been infected by a baculovirus that encodes said protein, although without limitation thereto.

It will be further understood that, as used throughout this specification, “increased” or “enhanced” baculovirus yield in a host cell that has been engineered according to the present invention, is relative to one or more corresponding host cells that have not been engineered according to the invention.

In some embodiments, the methods of the abovementioned aspects may be suitable for increasing or enhancing recombinant protein expression from a baculovirus in the host cell. It will be understood that, as used throughout this specification, “increased” or “enhanced” recombinant protein expression from a baculovirus in a host cell that has been engineered according to the present invention, is relative to recombinant protein expression from the baculovirus in one or more corresponding host cells that have not been engineered according to the invention.

It will be appreciated that engineering a host cell to express one or more of a p35, p35-trunc, p35-trunc-tail, and p49 protein may be performed by any suitable means. In some preferred embodiments, said engineering includes the step of transfecting a host cell with a genetic construct of the invention, as hereinabove described. Alternatively or additionally, said engineering may include infecting the host cell with a virus encoding a p35, p35-trunc, p35-trunc-tail, and/or p49 protein. In preferred such embodiments, said virus is a baculovirus. Suitably, said virus may be a different virus from the virus for which yield is increased or enhanced according to the above aspects.

In certain embodiments, for the purpose of engineering a host cell to express p35, p35-trunc, p35-trunc-tail, and/or p49, the host cell is infected with a virus for which the host cell is not a natural host. It will be appreciated that, in this context, ‘infection’ may refer to a partial or incomplete interaction in which no substantial replication of the virus occurs, but during which proteins encoded by the virus are expressed.

By way of non-limiting example, with reference to the examples, it will be appreciated that engineering of HzAM1 to express p35 may be possible by infection of HzAM1 with AcMNPV. It will be understood that HzAM1 is not a natural host for AcMNPV and that infection of HzAM1 with AcMNPV is a partial or incomplete interaction in which no substantial replication of AcMNPV occurs, but during which p35 encoded by AcMNPV may be expressed in HzAM1. As set forth in the examples, infection of HzAM1 with AcMNPV prior to infection with HearNPV (referred to as ‘pre-infection’ with AcMNPV) resulted in increased accumulation of HearNPV, which is hypothesized to have occurred due to expression of p35 from AcMNPV in the pre-infected HearNPV cells.

### Host Cells Expressing Combinations of Proteins

Certain aspects of the invention provide methods for producing an isolated host cell expressing particular combinations of p35, p35-trunc, p35-trunc-tail, and/or p49 proteins, as hereinabove described.

One such aspect is directed to a method for producing an isolated host cell expressing more than one of a p35, p35-trunc, and p35-trunc-tail protein.

Suitably, a host cell produced according to these aspects can facilitate increased or enhanced baculovirus yield. In some embodiments, said host cells can facilitate increased or enhanced recombinant protein expression from a baculovirus.

The inventors postulate that the expression of more than one of p35, p35-trunc, and p35-trunc-tail, may be more effective for inhibiting host cell defences in response to baculovirus infection than one of p35, p35-trunc, and p35-trunc-tail alone, and that this may therefore lead to an increased or enhanced yield of said baculovirus in a host cell expressing more than p35, p35-trunc, or p35-trunc-tail.

In an embodiment of this aspect, a host cell capable of expressing p35 is engineered to express one or more of p35-trunc, and p35-trunc-tail.

Another such aspect is directed to a method for producing an isolated host cell expressing (i) at least one of a p35, p35-trunc, and p35-trunc-tail protein; and (ii) a p49 protein.

The inventors postulate that the expression of (i) at least one of a p35, p35-trunc, and p35-trunc-tail protein; and (ii) a p49 protein, may be more effective for inhibiting host cell defences in response to baculovirus infection than (i) or (ii) alone, and this may therefore lead to an increased or enhanced yield of said baculovirus in a host cell expressing (i) and (ii).

In an embodiment of this aspect, a host cell capable of expressing a p35 protein is engineered to express a p49 protein.

In another embodiment of this aspect, a host cell capable of expressing a p49 protein is engineered to express one or more of a p35 protein, a p35-trunc protein, and a p35-trunc-tail protein.

In certain embodiments of the abovementioned aspects, the host cell may be infected by a baculovirus.

In a related aspect, the invention provides an isolated host cell suitable for infection by a baculovirus, wherein said host cell is capable of expressing more than one of a p35 protein, a p35-trunc protein, and a p35-trunc-tail protein.

In another related aspect, the invention provides an isolated host cell suitable for infection by a baculovirus, wherein said host cell is capable of expressing:

(i) at least one of a p35 protein, a p35-trunc protein, and a p35-trunc-tail protein; and

(ii) a p49 protein.

It will be understood that, in embodiments of the abovementioned aspects wherein a host cell is engineered to express multiple proteins, the relative order in which the host cell is engineered to express each of said proteins may be varied. Furthermore, in some such embodiments, the host cell may be engineered to express any combination of said proteins simultaneously. It will be further appreciated that provided according to this aspect, without limitation, are embodiments wherein the host cell may be suitable for one or more of:

(a) increasing the yield of a baculovirus in a host cell wherein the baculovirus does not normally encode a p35 protein; and

(b) increasing the yield of a baculovirus in a host cell wherein the baculovirus does not normally encode a p49 protein; and

(c) increasing the yield of a recombinant protein from a recombinant baculovirus in a host cell wherein the baculovirus does not normally encode a p35 protein; and

(d) increasing the yield of a recombinant protein from recombinant baculovirus in a host cell wherein the baculovirus does not normally encode a p49 protein.

In certain embodiments, an isolated, engineered host cell according to this aspect may be infected by a baculovirus.

In one embodiment, said baculovirus does not normally encode a p35 protein.

In another embodiment, said baculovirus does not normally encode a p49 protein.

In certain embodiments, said baculovirus is a recombinant baculovirus.

It will be understood that the host cell may be engineered to increase yield of a baculovirus, whether or not the baculovirus is a recombinant baculovirus that encodes a protein for expression in the host cell.

Preferably, a host cell according to the present invention is an arthropod cell.

More preferably, said host cell is an insect cell.

In preferred embodiments of the invention wherein said host cell is an insect cell, said host cell is selected from the group consisting of: Sf and cell clones and/or cell populations derived from these cells, e.g. Sf9 and Sf21 cells; Hzea and cell clones and/or cell populations derived from these cells, e.g. HzAM1; Tni and cell clones and/or cell populations derived from these cells, e.g. Hi5 and TN368; Px and cell clones and/or cell populations derived from these cells, Ag and cell clones and/or cell populations derived from these cells, e.g. saUFL-AG-286 and UFL-AG-286; and Heteronychus arator and cell clones and/or cell populations derived from these cells, e.g. DSIR-HA-1179

With regard to aspects of the invention relating to a baculovirus wherein said baculovirus does not encode a p35 protein, preferably said baculovirus is selected from the group consisting of: AgMNPV; HearNPV; SfMNPV; Buzura suppressaria nucleopolyhedrovirus; Choristoneura fumiferana DEF multiple nucleopolyhedrovirus; Choristoneura fumiferana multiple nucleopolyhedrovirus; Chrysodeixis chalcites nucleopolyhedrovirus; Clanis bilineata nucleopolyhedrovirus; Ectropis obliqua nucleopolyhedrovirus; Epiphyas postvittana nucleopolyhedrovirus; Euproctis pseudoconspersa nucleopolyhedrovirus; Lymantria dispar multiple nucleopolyhedrovirus; Mamestra brassicae multiple nucleopolyhedrovirus; Mamestra configurata nucleopolyhedrovirus A; Mamestra configurata nucleopolyhedrovirus B; Orgyia pseudotsugata multiple nucleopolyhedrovirus; Spodoptera exigua multiple nucleopolyhedrovirus; Spodoptera littoralis nucleopolyhedrovirus; Trichoplusia ni single nucleopolyhedrovirus; Wiseana signata nucleopolyhedrovirus; Adoxophyes orana granulovirus; Artogeia rapae granulovirus; Choristoneura fumiferana granulovirus; Cryptophlebia leucotreta granulovirus; Cydia pomonella granulovirus; Harrisina brillians granulovirus; Helicoverpa armigera granulovirus; Lacanobia oleracea granulovirus; Phthorimaea operculella granulovirus; Plodia interpunctella granulovirus; Plutella xylostella granulovirus; Pseudalatia unipuncta granulovirus; Trichoplusia ni granulovirus; Xestia c-nigrum granulovirus; Culex nigripalpus nucleopolyhedrovirus; Neodiprion lecontei nucleopolyhedrovirus; and Neodiprion sertifer nucleopolyhedrovirus, inclusive of recombinant baculoviruses derived from said baculoviruses.

In one preferred embodiment said baculovirus is HearNPV.

In another preferred embodiment said baculovirus is SfMNPV.

In another preferred embodiment said baculovirus is AgMNPV.

In preferred embodiments relating to HearNPV, the host cell is a Hzea cell line or a derivative thereof. Preferably, said host cell is HzAM1, or a derivative thereof.

In preferred embodiments relating to SfMNPV, the host cell is an Sf cell line or a derivative thereof. Preferably, said host cell is Sf9 or Sf21, or derivatives thereof.

In preferred embodiments relating to AgMNPV, the host cell is an Ag cell line, or a derivative thereof. Preferably, said host cell line is saUFL-AG-286 (as set forth in Micheloud et al. 2011, J Virol. Methods 178 106) or UFL-AG-286, or derivatives thereof.

With regard to aspects of the invention relating to a baculovirus wherein said baculovirus does not encode a p49 protein, preferably said baculovirus is selected from the group consisting of: HearNPV; SfMNPV; PxMNPV; AgMNPV; AcMNPV; Adoxophyes honmai nucleopolyhedrovirus; Agrotis ipsilon multiple nucleopolyhedrovirus; Agrotis segetum nucleopolyhedrovirus; Antheraea pernyi nucleopolyhedrovirus; Bombyx mori nucleopolyhedrovirus; Buzura suppressaria nucleopolyhedrovirus; Choristoneura fumiferana DEF multiple nucleopolyhedrovirus; Choristoneura fumiferana multiple nucleopolyhedrovirus; Choristoneura rosaceana nucleopolyhedrovirus; Chrysodeixis chalcites nucleopolyhedrovirus; Clanis bilineata nucleopolyhedrovirus; Ectropis obliqua nucleopolyhedrovirus; Epiphyas postvittana nucleopolyhedrovirus; Euproctis pseudoconspersa nucleopolyhedrovirus; Hyphantria cunea nucleopolyhedrovirus; Lymantria dispar multiple nucleopolyhedrovirus; Mamestra brassicae multiple nucleopolyhedrovirus; Mamestra configurata nucleopolyhedrovirus A; Mamestra configurata nucleopolyhedrovirus B; Maruca vitrata nucleopolyhedrovirus; Orgyia pseudotsugata multiple nucleopolyhedrovirus; Spodoptera exigua multiple nucleopolyhedrovirus; Thysanoplusia orichalcea nucleopolyhedrovirus; Trichoplusia ni single nucleopolyhedrovirus; Wiseana signata nucleopolyhedrovirus; Adoxophyes orana granulovirus; Artogeia rapae granulovirus; Choristoneura fumiferana granulovirus; Cryptophlebia leucotreta granulovirus; Cydia pomonella granulovirus; Harrisina brillians granulovirus; Helicoverpa armigera granulovirus; Lacanobia oleracea granulovirus; Phthorimaea operculella granulovirus; Plodia interpunctella granulovirus; Plutella xylostella granulovirus; Pseudalatia unipuncta granulovirus; Trichoplusia ni granulovirus; Xestia c-nigrum granulovirus; Culex nigripalpus nucleopolyhedrovirus; Neodiprion lecontei nucleopolyhedrovirus; Neodiprion sertifer nucleopolyhedrovirus, inclusive of recombinant baculoviruses derived from said baculoviruses (e.g. rAcMNPV).

In one preferred embodiment said baculovirus is HearNPV.

In another preferred embodiment said baculovirus is SfMNPV.

In another preferred embodiment said baculovirus is PxMNPV.

In another preferred embodiment said baculovirus is AgMNPV.

In another preferred embodiment said baculovirus is AcMNPV.

In another preferred embodiment said baculovirus is rAcMNPV.

In preferred embodiments relating to HearNPV, the host cell is a Hzea cell line or a derivative thereof. Preferably, said host cell is HzAM1, or a derivative thereof.

In preferred embodiments relating to SfMNPV, the host cell is an Sf cell line or a derivative thereof. Preferably, said host cell is Sf9 or Sf21, or derivatives thereof.

In preferred embodiments relating to PxMNPV, the host cell is a Tni cell line or a derivative thereof. Preferably, said host cell is Hi5 and TN368, or derivatives thereof.

In preferred embodiments relating to AgMNPV, the host cell is an Ag cell line, or a derivative thereof. Preferably, said host cell line is saUFL-AG-286 (as set forth in Micheloud et al. 2011, J Virol. Methods 178 106) or UFL-AG-286, or a derivative thereof.

In certain preferred embodiments relating to AcMNPV or rAcMNPV the host cell is an Sf cell line or a derivative thereof. Preferably, said host cell is Sf9 or Sf21, or derivatives thereof.

In certain other preferred embodiments relating to AcMNPV or rAcMNPV the host cell is a Tni cell line or a derivative thereof. Preferably, said host cell is Hi5 and TN368, or derivatives thereof.

### Baculovirus Yield

As hereinabove described, baculovirus yield may be increased or enhanced by expressing one or more of p35, p35-trunc, p35-trunc-tail, and p49.

Baculovirus yield in a host cell may be measured by any suitable method selected from the range of methods known to those skilled in the art. For example, the skilled person is directed to Flint et al. PRINCIPLES OF VIROLOGY: PATHOGENESIS AND CONTROL (3^{rd} Edition; ASM Press, 2008), incorporated herein by reference, in particular Volume I, Part 1, Chapter 2.

In certain preferred embodiments, baculovirus yield is measured by the number of “occlusion bodies” (“OBs”) per baculovirus-infected host cell.

In certain embodiments, increased baculovirus yield in an engineered host cell may be an increase in OBs per baculovirus-infected host cell of greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, or greater than 100%, relative to one or more corresponding baculovirus-infected host cells that have not been engineered according to the invention.

As will be understood by one skilled in the art, baculovirus yield in a host cell may be correlated to cell size post-infection. Therefore, in certain embodiments an increased baculovirus yield in an engineered host cell according to the invention may be measured as a greater increase in cell size post-infection, relative to one or more corresponding host cells that have not been engineered according to the invention.

In certain embodiments, increased baculovirus yield in an engineered host cell may be an increase in cell size of a baculovirus-infected host cell of greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, or greater than 100%, relative to one or more corresponding baculovirus-infected host cells that have not been engineered according to the invention.

It will be further understood that an increase in baculovirus yield in a host cell may be correlated to an increase in polyhedrin protein accumulation, or accumulation of a nucleic acid encoding polyhedrin protein, in the host cell.

In certain embodiments, increased baculovirus yield in an engineered host cell may be an increase in polyhedrin protein accumulation, or an increase in accumulation of a nucleic acid encoding a polyhedrin protein, in a baculovirus-infected host cell of greater than 10%, greater than 20%, greater than 30%, greater than 40%, greater than 50%, greater than 60%, greater than 70%, greater than 80%, greater than 90%, or greater than 100%; or greater than 150%, greater than 200%, greater than 250%, greater than 300%, greater than 350%, greater than 400%, greater than 450%, or greater than 500%, relative to one or more corresponding baculovirus-infected host cells that have not been engineered according to the invention.

### Recombinant Protein Expression

As hereinabove described, in some embodiments, increased or enhanced baculovirus yield may facilitate increased or enhanced recombinant protein expression by the baculovirus.

With regard to aspects of the invention relating to the expression of recombinant proteins, it will be appreciated by the skilled person that methods for the engineering of recombinant baculoviruses, and for the expression and purification of recombinant proteins from recombinant baculoviruses in host cells, are well known in the art. For example, the skilled person is referred to Kost et al., 2005, Nat. Biotechnol. 23 567; and GUIDE TO BACULOVIRUS EXPRESSION SYSTEMS (BEVS) AND INSECT CELL CULTURE TECHNIQUES (Invitrogen/Life Technologies; Instruction Manual; http://tools.lifetechnologies.com/content/sfs/manuals/bevtest.pdf) and references therein, incorporated herein by reference.

The one or more recombinant proteins expressed from a baculovirus according to these aspects of the invention may be any suitable protein(s). Non-limiting examples of recombinant proteins that may be suitable for expression from a baculovirus include pharmaceutical and/or diagnostic proteins, e.g. vaccines, hormones, and antibodies; proteins used in food production, e.g. proteases such as amylases, phytases, and pectinases; biopesticides, e.g. insect toxins; and biocatalysts, which may have application in the production of fine chemicals, bioremediation, biosensor technology, and medicinal chemistry.

Measurement of the level of expression of a recombinant protein in a host cell according to the invention may be performed using any of the range of methods known to those skilled in the art.

In one embodiment, recombinant protein expression can be detected by an antibody specific for the recombinant protein:

(i) in an ELISA such as described in Chapter 11.2 of CURRENT PROTOCOLS IN MOLECULAR BIOLOGY Eds. Ausubel et al. (John Wiley & Sons Inc. NY, 1995) which is herein incorporated by reference; or

(ii) by Western blotting and/or immunoprecipitation such as described in Chapter 12 of CURRENT PROTOCOLS IN PROTEIN SCIENCE 5 Eds. Coligan et al. (John Wiley & Sons Inc. NY, 1997), which is herein incorporated by reference.

In certain other embodiments, the expression of a recombinant protein may be assessed by measuring the expression of a transcript encoding the recombinant protein. Methods of measuring the expression of a transcript are well known in the art. For example, the expression of a transcript encoding a recombinant protein may be performed by Northern blotting, or by real-time reverse transcription PCR or qRT-PCR, as herein described.

### Antibodies

Also provided according to the invention are anti-p35-trunc-tail and anti-p35-trunc antibodies or antibody fragments binding to or raised against a protein comprising the amino acid sequence set forth in SEQ ID NO:3 or SEQ ID NO:4.

Antibodies of the invention may be polyclonal or monoclonal. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with a polypeptide, fragment, variant or derivative of the invention. For example, polyclonal antibodies may be prepared for example by injecting a polypeptide, fragment, variant or derivative of the invention into a production species, which may include mice, rabbits or goats, to obtain polyclonal antisera. Methods of producing polyclonal antibodies are well known to those skilled in the art. Exemplary protocols that may be used are described for example in Coligan et al., CURRENT PROTOCOLS IN IMMUNOLOGY, supra, and in Harlow & Lane, 1988, supra.

In lieu of the polyclonal antisera obtained in the production species, monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the polypeptides, fragments, variants or derivatives of the invention.

The invention also includes within its scope antibodies that comprise Fc or Fab fragments of the polyclonal or monoclonal antibodies referred to above. Alternatively, the antibodies may comprise single chain Fv antibodies (scFvs) against the peptides of the invention. Such scFvs may be prepared, for example, in accordance with the methods described respectively in U.S. Pat. No. 5,091,513, European Patent No 239,400 or the article by Winter & Milstein, 1991, Nature 349 293, which are incorporated herein by reference.

In order that the invention may be readily understood and put into practical effect, particular preferred embodiments will now be described by way of the following non-limiting examples.

### EXAMPLES

### Example 1

### Hzea Cells Transiently Expressing p35-trunc-Tail

### Materials and Methods

Transient Transfection of HzAM1 Cells with p35-trunc-Tail

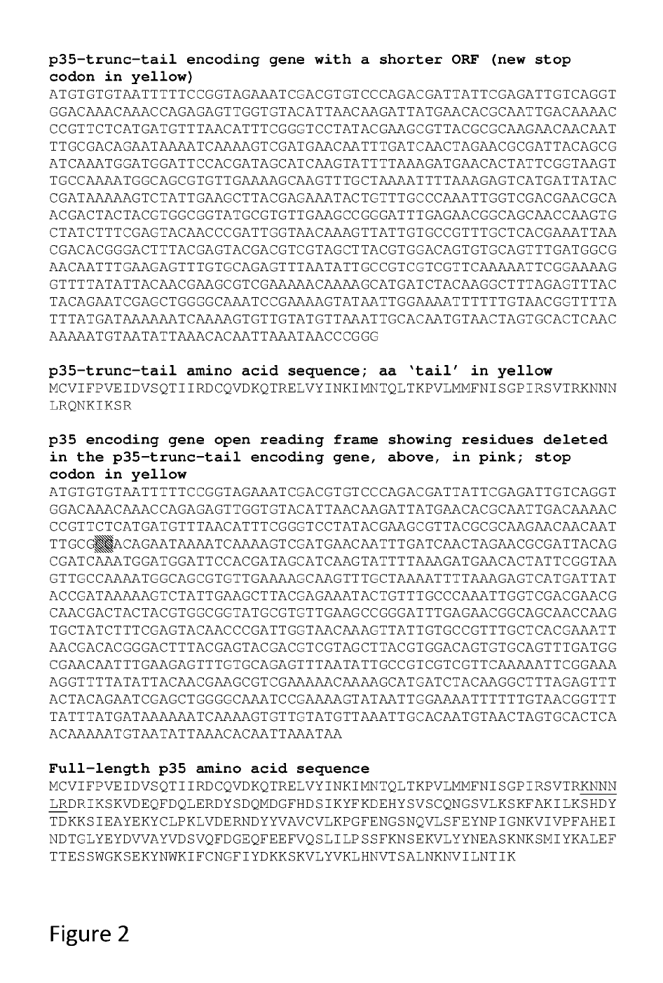

The p35-trunc-tail gene, as set forth in FIG. 2 and SEQ ID NO:9, which is a variant of the p35 gene from Autographa californica multiple nucleopolyhedrovirus (AcMNPV), was transfected into HzAM1 cells in pIZ/V5-His under the control of a baculovirus immediate early-1 (ie-1) promoter, designed to be active in insect cells. Transfected cells were allowed to grow for 60 h (10 ml cultures in 50 ml culture tubes, SF900III medium, 27° C., 250 rpm) without a selective agent, prior to infection as described below.

Infection of Control HzAM1 Cells and p35-trunc-Tail Transfected HzAM1 Cells by HearNPV

After 60 h growth, transiently p35-trunc-tail -transfected HzAM1 cells, and control HzAM1 cells, were infected with HearNPV. Two independent experiments were conducted; the first experiment was conducted in triplicate (3 transfected and 3 control cultures), while the second experiment to confirm the first result was conducted in duplicate.

Quantification of HearNPV Virus Yield from Control and p35-trunc-Tail Transfected HzAM1 Cells

p35-trunc-tail transiently transfected HzAM1 cells at 60 h posttransfection and control HzAM1 cells were diluted to 5×10⁵ cells/ml with SF900III medium and infected with a HearNPV virus at a multiplicity of infection of 10 PFU/ml (10 ml culture volumes in 50 ml culture tubes or 20 ml culture volumes in 125 ml shakers on an orbital shaker operated at 250 rpm). Occlusion body (OB) counts were conducted from infected cultures at 7 days postinfection.

Quantification of Size of HearNPV-Infected Control and p35-trunc-Tail—Transfected HzAM1 Cells

The cell size of infected cultures was determined at 2 days post-infection using a Multisizer 4 Particle Analyzer.

### Results

Baculovirus Polyhedrin Accumulation in p35-trunc-Tail Transfected and Control HzAM1 Cells

Polyhedrin protein expression in infected, transiently transfected p35-trunc-tail HzAM1 cells compared with infected, non-transfected HzAM1 control cells, derived from SDS PAGE polyhedrin densitometry studies is set forth in Table 1. Two-tail T test analyses identified the statistical significance of the difference in the mean polyhedrin level between the transfected and non-transfected cases. This analysis can be used as a rough comparison with the OB/cell yield ratio. Note that OBs are produced in varying sizes, and so the ratio of physical particles versus polyhedrin protein density may not match exactly.

OB/Cell of HearNPV in p35-trunc-Tail Transfected and Control HzAM1 Cells

The average OB/cell for the control experiments (5 replicates total) was 396 while for the transfected cultures the average OB/cell (5 replicates total) was 691, an ˜70% increase. The control yields were consistent with optimal yields that have been observed for such infected cultures over the past decade.

Increase in Cell Size of p35-trunc-Tail Transfected HzAM1 Cells Infected with HearNPV

The yield/cell of OB has been well correlated to an increase in cell size post-infection. Typically, HzAM1 cells double in size when infected by HearNPV, as compared to non-infected controls. The infected p35-trunc-tail transfected cells increased in size three fold compared to control non-infected cells (an ˜50% greater increase in size). This is further evidence that the infection was more robust, i.e. the infected cells accumulated a greater yield of virus, for the transfected cells.

### Conclusions

We have demonstrated that HearNPV yield in HzAM1 cells transiently transfected to express p35-trunc-tail was substantially increased, relative to control HzAM1 cells that do not express p35-trunc-tail. As will be understood by the skilled person, HearNPV does not normally encode p35. It will therefore be understood that these results demonstrate that the expression of p35-trunc-tail in baculovirus-infected host cells can lead to a substantial increase in baculovirus yield, wherein the baculovirus does not normally encode p35. With reference to Example 4, below, it will be further appreciated that the same or similar results may well be achieved using wild type p35, and/or with at least some other suitable p35 fragments, such as p35-trunc.

In light of the surprising results presented here, it appears that the expression of p35, p35-trunc, and/or p35-trunc-tail in a host cell may be particularly effective for increasing the yield of a baculovirus in the host cell, wherein the baculovirus does not normally encode a p35 protein. It may be that host cell defences against a baculovirus that does not normally encode a p35 protein are relatively ineffective against the accumulation of a baculovirus in the presence of p35, p35-trunc, and/or p35-trunc-tail.

### Example 2

### Hzea Cells Stably Expressing p35-trunc-Tail

### Material and Methods

Zeocin Selection of p35-trunc-Tail Cells; Establishment of Stable p35-trunc-Tail Stocks

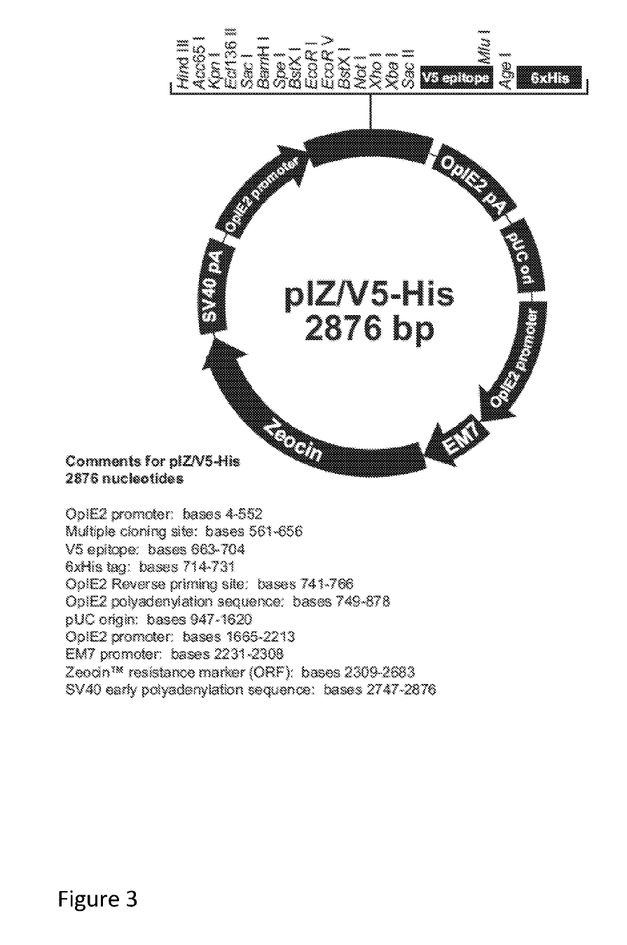

HzAM1 cells were transfected with the p35-trunc-tail gene using the pIZN5-His vector (set forth in FIG. 3), comprising a p35-trunc-tail expression cassette. As will be evident from FIG. 3, pIZ/V5-His features a Zeocin™ resistance gene and facilitates selection of stably transfected cells using Zeocin.

As set forth below, experiments were performed to identify an ideal Zeocin level for selecting stably transfected cells. Such an ideal level was considered to be one that kills off non-transfected cells within 2 weeks. In this respect, if excessively high levels are used there is a chance the transfected cells will be killed off before cells are able to integrate the resistance gene into their genome and express it at sufficient levels to counter the Zeocin. However if levels of Zeocin are too low then elimination of non-transfected cells may not be achieved.

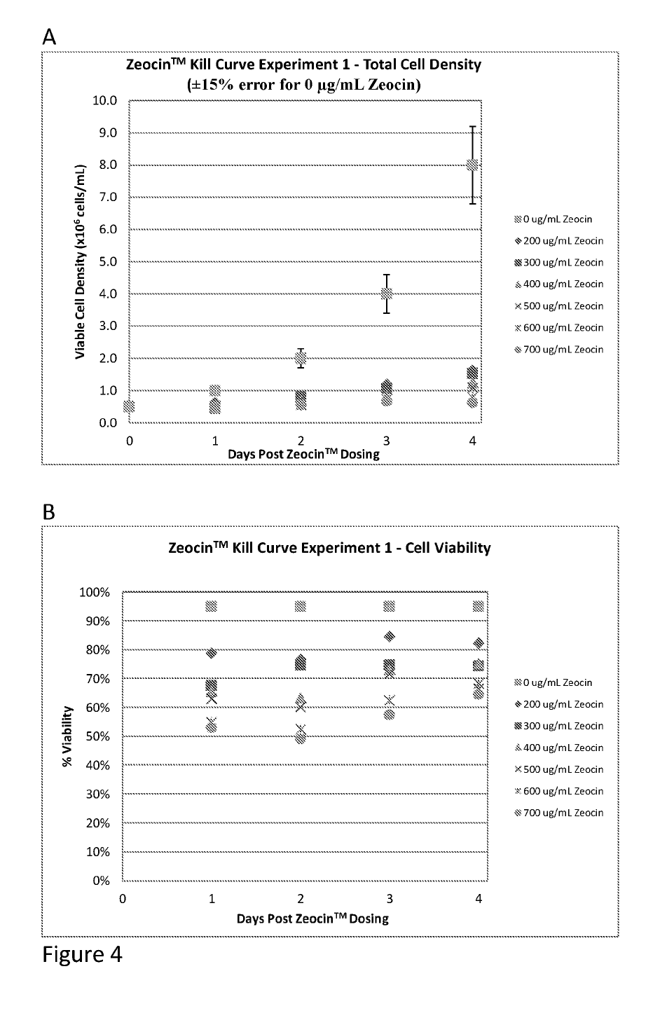

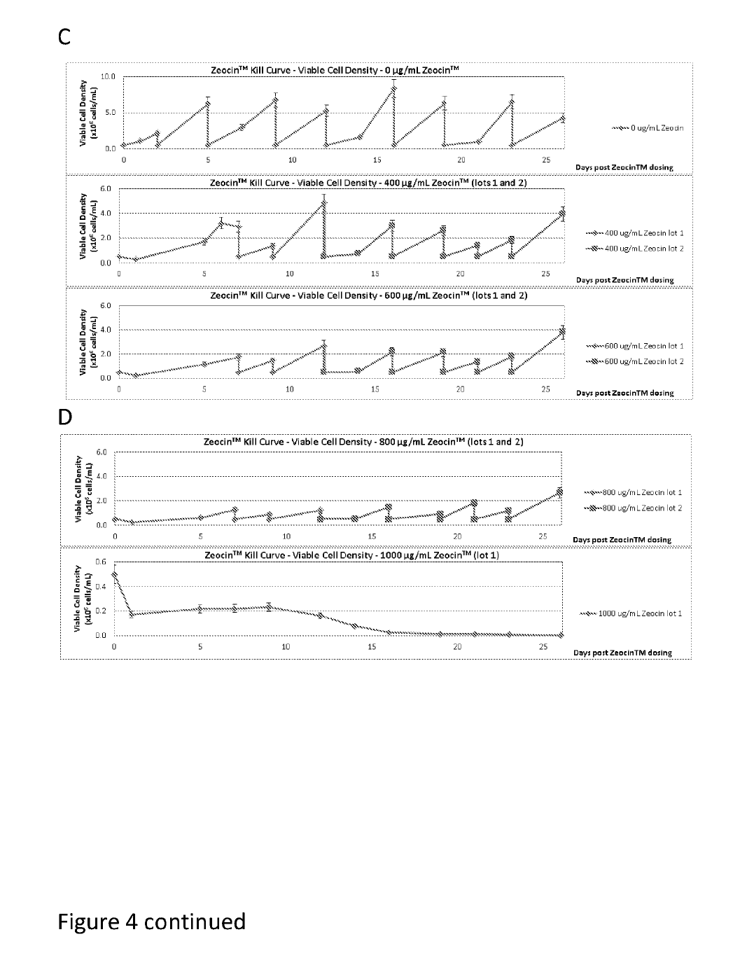

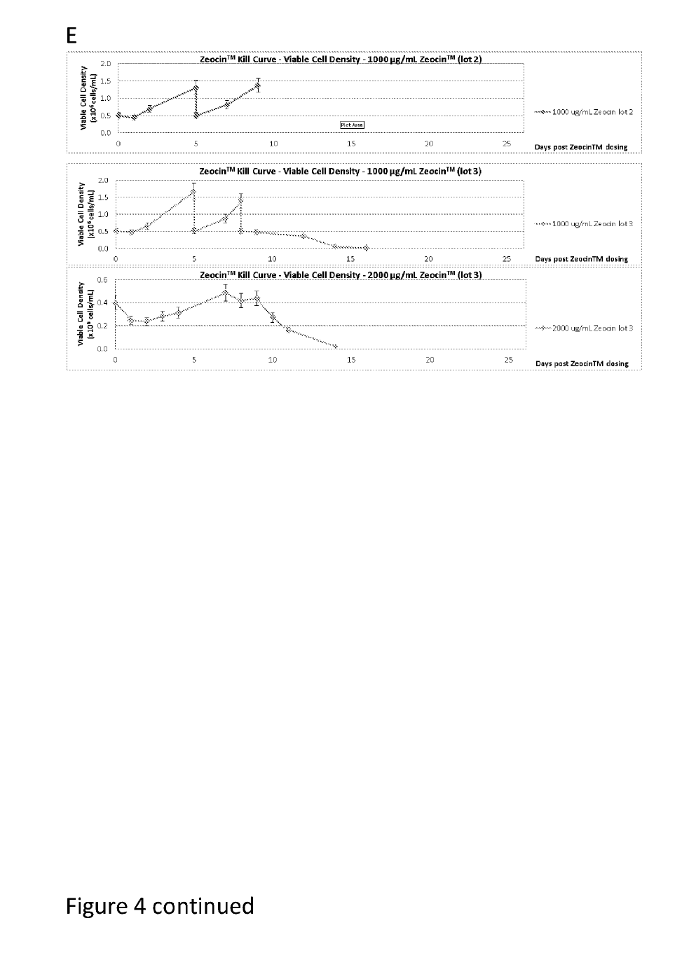

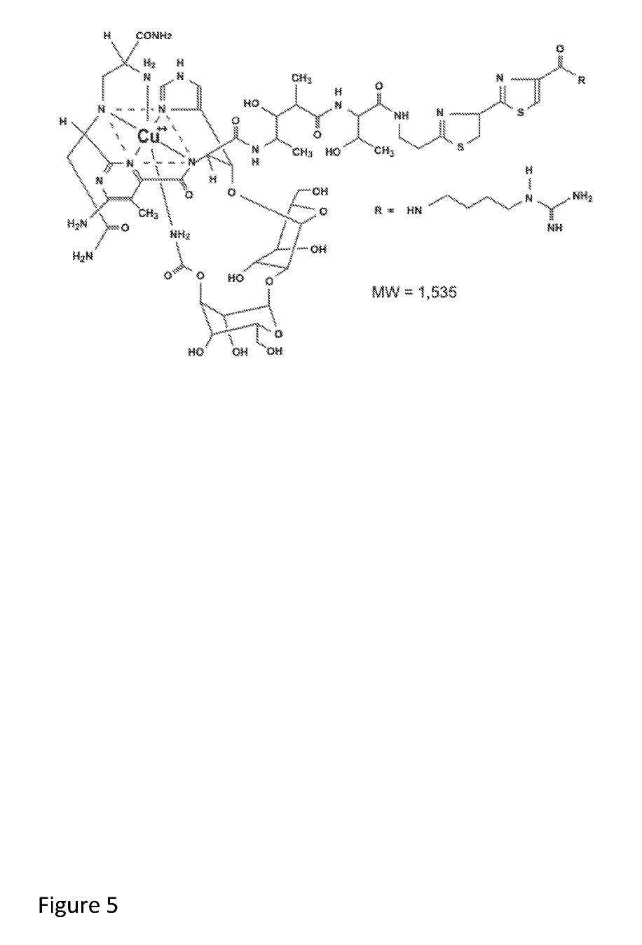

A series of kill curves were undertaken to determine the optimal concentrations and a dosing regimen was established for selection of Zeocin-resistant HzAM1 cells in SF900III (Invitrogen Life Technologies, 2012). Note that all Zeocin Kill Curve analyses were performed using suspension culture to maximise potential cell growth rates and viabilities for the Zeocin treatments, and to allow accurate monitoring of cell growth and viabilities throughout the Zeocin treatment procedures. The results of some of these analyses are set forth in FIG. 4.

Based on the data presented in FIG. 4 it was concluded that 2,000 μg/ml was necessary to kill off non transfected cells in a reasonable time period.

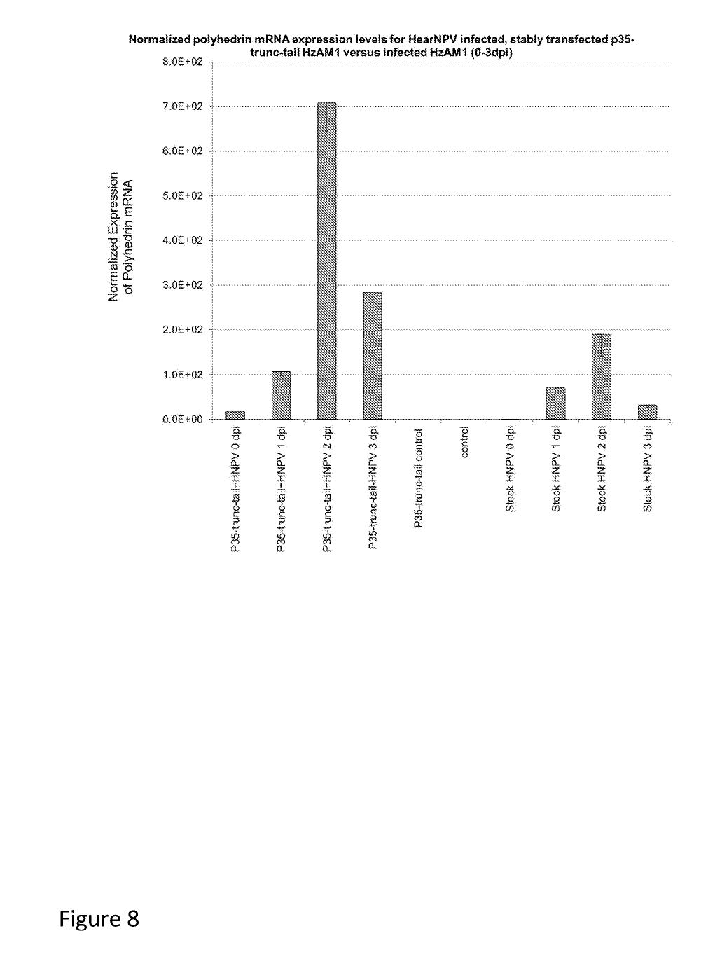

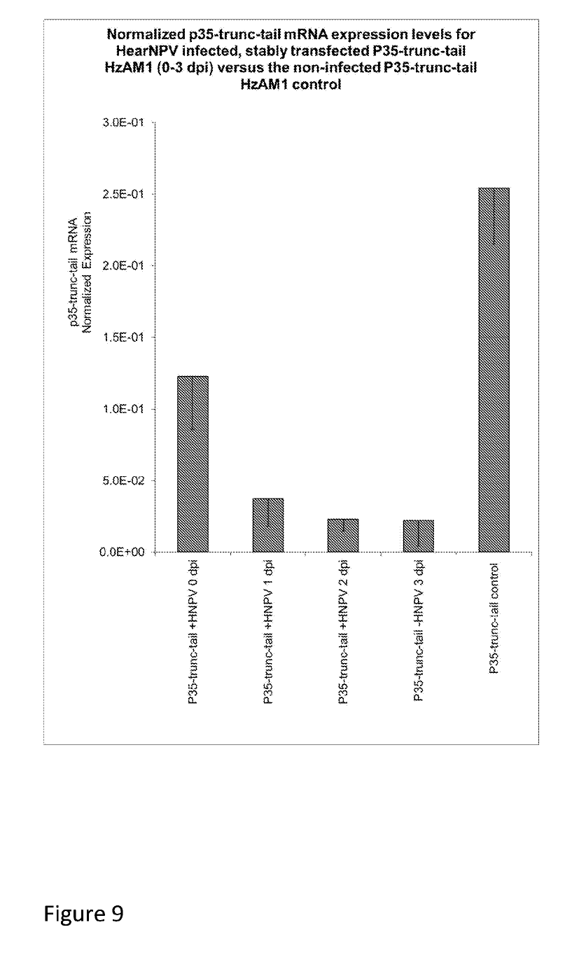

The stably transfected cells produced therefore were selected over a 3 month period of being exposed to 400, 600, 1,000 and eventually 2,000 ug/ml of Zeocin. The stably transfected/infected data shown below in Tables 2 and 3 was obtained using p35-trunc-tail transfected cells after they had been exposed to 1,000 ug/ml of Zeocin for a number of weeks. The cells used for the stable transfected/infected experiment were sourced from a frozen stock of stably transfected cells (Freeze F-76) and these were the cells used for the p35-trunc-tail PCR analysis shown in FIG. 8 and FIG. 9.

Subsequently the transfected cells used for the infection experiment were exposed to 2,000 ug/ml of Zeocin and were shown to survive and grow well even at these very high levels of Zeocin.

Frozen stocks of the p35-trunc-tail transfected cells exposed to 600, 800, 1,000 and 2,000 ug/ml of Zeocin were made. For freezing and for infection experiments the cells needed to be weaned off Zeocin over a period of 2 weeks. This is because cells when frozen are exposed to DMSO and are unlikely to freeze/thaw successfully if exposed to Zeocin as well during the freeze/thaw process. When infected it is not possible to have Zeocin present as this could mutate the virus DNA.

Baculovirus Infection of Stably Transfected p35-trunc-Tail Cells, and Control Cells

Stably p35-trunc-tail transfected HzAM1 cells, transiently p35-trunc-tail transfected and control HzAM1 cells (for comparison) were infected at 5×10⁵ cells/ml in shaker flasks (in triplicate) with HearNPV. This low cell density was designed to ensure that the cells were infected under optimal early exponential phase conditions, and will lead to maximum cell specific occlusion body (OB) yields. Infections were conducted using a high multiplicity of infection, MOI, of virus to achieve minimal cell growth post infection.

### Results

Growth Rate of Non-Infected Cells

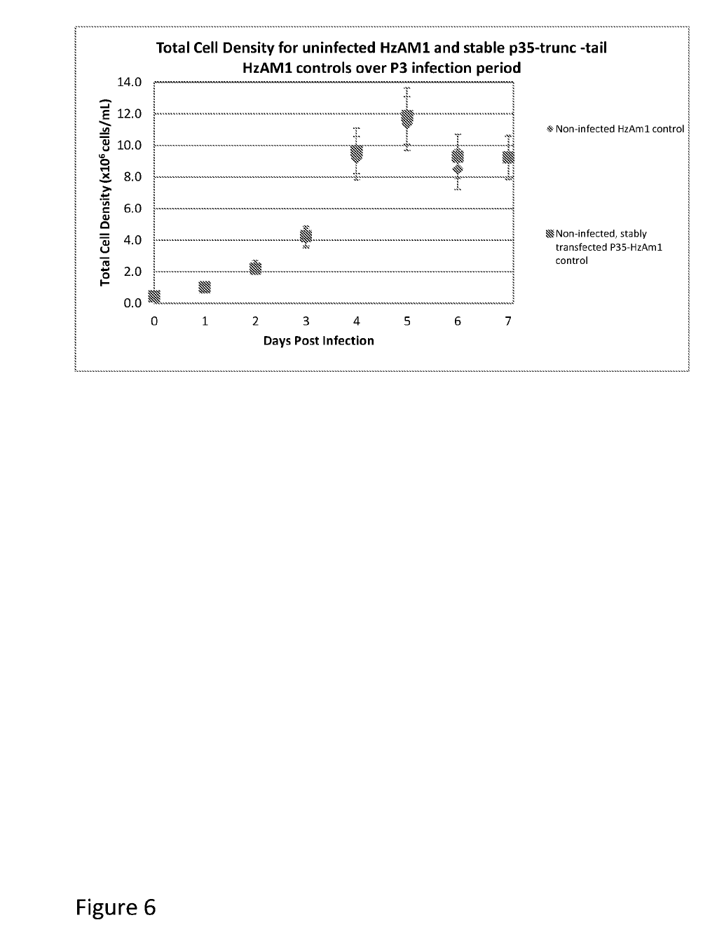

The growth rate of non-infected (i) HzAM1 cells and (ii) stably transfected p35-trunc-tail HzAM1 cells in SF900III media (Life Technologies) is set forth in FIG. 6. This figure shows that the cells used for the infections displayed normal exponential growth, when uninfected. The p35-trunc-tail transfected cells in this case had been weaned off Zeocin in preparation for the infection experiment; it was observed that the p35-trunc-tail transfected cells grew at a slightly reduced growth rate when in the presence of very high levels of Zeocin (1,000-2,000 ug/ml).

Growth Rate of Infected Cells

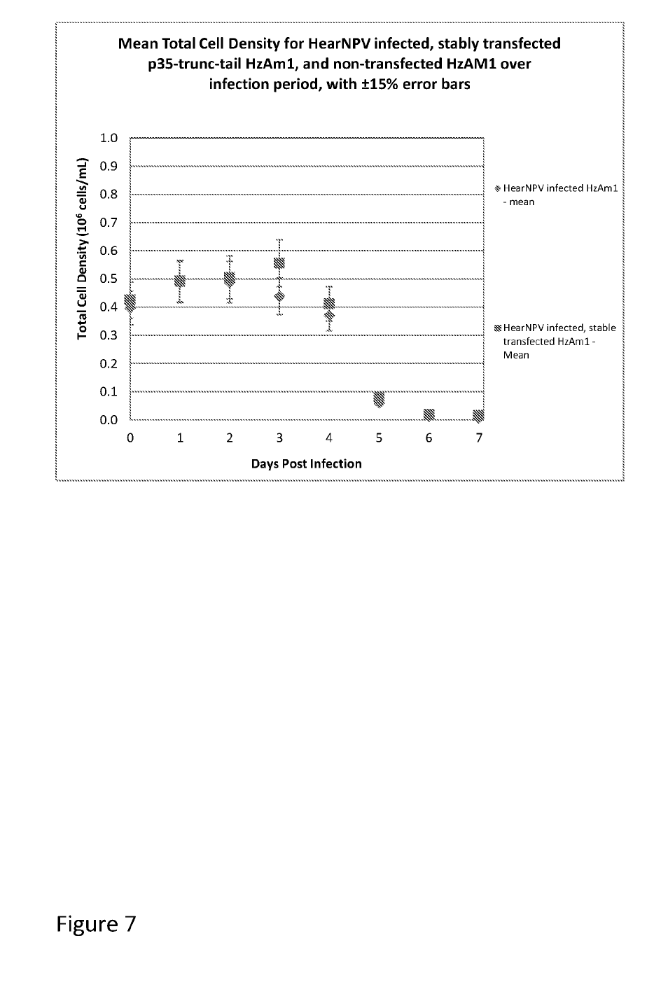

The growth rate of HearNPV infected HzAM1 cells and stably p35-trunc-tail transfected HzAM1 cells in SF900III media (Life Technologies) is set forth in FIG. 7. This figure shows that p35-trunc-tail transfected HzAM1 cells displayed a slightly extended period of cell viability before succumbing to the virus infection.

Occlusion Body Yield in Transfected Cells

Occlusion Body/Cell yield of stably transfected p35-trunc-tail HzAM1 cells and non-transfected HzAM1 (and transiently transfected cells for comparison) was measured and compared. Results are set forth in Tables 2 and 3.

These results demonstrate that the increase in OB yield in stably transfected p35-trunc-tail HzAM1 cells as compared to non-transfected cells was substantial, and comparable to the increase observed in transiently transfected p35-trunc-tail HzAM1 cells. Specifically, the stably transfected cells were found to produce ˜1.7× the OB/cell yield of the control HzAM1 cells, which matches the result seen for the transiently transfected HzAM1 cells in the experiment conducted for this example (Table 3), and the results of the independent experiments conducted for Example 1, as set forth above.

Baculovirus Polyhedrin Expression in Transfected and Control HzAM1 Cells

Normalized expression levels for polyhedrin mRNA in HearNPV infected stably transfected p35-trunc-tail HzAM1, and HearNPV infected non-transfected HzAM1 (from 0-3 days post infection; dpi) was assessed using qRT-PCR, using a protocol as follows:

Total genomic RNA was extracted from cells by using aPhenol/guanidine-based QIAzol Lysis reagent and subsequently treated with DNase I before being used for reverse transcription. A total of 2 μg of RNA for each sample was reverse transcribed generating complementary DNA from RNA samples. qPCR with gene-specific primers was performed to determine their mRNA levels in different samples. For each qPCR Platinum SYBR Green Mix (Qiagen) with 2 μL of the first-strand cDNA reaction was used in a Rotor-Gene thermal cycler (Qiagen) under the following conditions: 95° C. hold for 30 s, then 40 cycles of 95° C. for 15 s, 50° C. for 15 s, and 72° C. for 20 s.

Results are set forth in FIG. 8. Variable errors (error bars) are calculated as the standard error of the mean of the normalised expression which is derived from technical and biological replicates. These results indicate a ˜3.5× increase in polyhedrin mRNA expression from HearNPV infected stably transfected p35-trunc-tail HzAm1 cells compared with infected non-transfected HzAM1 control cells at 2 dpi. Furthermore, polyhedrin mRNA expression peaked at 2 dpi and decreased at 3 dpi in both the stably transfected p35-trunc-tail HzAM1 cells and the HzAM1 control cells.

p35-trunc-Tail Expression in HearNPV-Infected and Non-Infected p35-trunc-Tail HzAM1 Cells

The expression of mRNA encoding p35-trunc-tail was assessed using qRT-PCR in HearNPV infected and non-infected stably transfected p35-trunc-tail HzAM1 cells. The protocol used was as follows:

Total genomic RNA was extracted from cells by using aPhenol/guanidine-based QIAzol Lysis reagent and subsequently treated with DNase I before being used for reverse transcription. A total of 2 μg of RNA for each sample was reverse transcribed generating complementary DNA from RNA samples. qPCR with gene-specific primers was performed to determine their mRNA levels in different samples. For each qPCR Platinum SYBR Green Mix (Qiagen) with 2 μL of the first-strand cDNA reaction was used in a Rotor-Gene thermal cycler (Qiagen) under the following conditions: 95° C. hold for 30 s, then 40 cycles of 95° C. for 15 s, 50° C. for 15 s, and 72° C. for 20 s. qPCR primers targeting p35-trunc-tail were 5′-TGGATTCCACGATAGCATCA-3′ (Forward); and 5′-GACCAATTTGGGCAAACAGT-3′ (Reverse)

Results are set forth in FIG. 9. This data confirms that the stably transfected cells are expressing p35-trunc-tail. The PCR data suggests that expression of the p35-trunc-tail gene by the p35-trunc-tail transfected host cells is downregulated post infection.

### Discussion

Overall the data presented in this example establishes that the p35-trunc-tail stably transfected cells produce 1.7× the OB/cell yield of the control HzAM1 cells which matches the result seen for the transiently transfected HzAM1 cells. The PCR data confirms that the stably transfected cells are expressing p35-trunc-tail and show elevated levels of polyhedral mRNA post infection compared to that seen in infected control (non-transfected) HzAM1 cells. The PCR data suggests that expression of the p35-trunc-tail gene by the p35-trunc-tail transfected host cells is downregulated post-infection and so the p35-trunc-tail effect on yield appears likely to be due to residual p35-trunc-tail protein expressed prior to infection that persists for some time post infection.

In view of the results presented in Example 4, below, it will be appreciated that wild type p35 (and at least some other p35 fragments, such as p35-trunc) used in place of p35-trunc-tail may well achieve similar results as those described in this example.

### Example 3

### VSR Activity of p35

### Materials and Methods

Cells and Virus

Spodoptera frugiperda cell line (Sf9) was maintained in SF900-II serum free medium (Invitrogen) as a monolayer at 27° C. AcMNPV was amplified in Sf9 cells and budded viruses accumulated in the medium were used for inoculations. For AcMNPV infection, 2×10⁶ cells were infected at a multiplicity of infection (MOI) of 5 PFU/cell as described (King and Possee 1992) diluted in Sf900-II medium. An hour after incubation at 27° C., fresh medium was added to cells and incubated further at 27° C. Vero and NIH-3T3 cells were maintained as monolayers at 37° C. in RPMI-1640 medium with 5% fetal bovine serum (FBS).

Cloning of p35

The p35 gene was amplified from AcMNPV-infected Sf9 cells using specific forward and reverse primers bearing Sad and SacII restriction sites, respectively. Then, the amplified p35 gene was cloned into the pIZ/V5-His expression vector resulting in pIZ/p35. To create p35 mutant genes, different truncations were made at both termini of the gene. The first mutant construct was made by truncation at the 3′ end (654-900) using Sspl restriction enzyme on pIZ/p35 followed by self-ligation producing the mutant gene pIZ/p35Δ1. The other two p35 mutants were made by deletion of 30 nt (pIZ/p35Δ2) and 90 nt at the 5′ end pIZ/p35Δ3), using specific forward primers bearing Sad restriction site and ATG starting codon. p35 orf was also cloned into the mammalian expression vector pEGFP-N1 using BglII and XmaI restriction sites. Expression of p35 from the constructs was confirmed by RT-PCR using a pair of primers to the middle of p35 (p35-mid F & R; Table 4).

Electrophoresis and Western Blotting

To visualize the levels of specific proteins (prohibitin, GFP, gp64), protein samples from whole cells (same number of cells) were run on a denaturing 12% SDS-PAGE gel and Western blotting was carried out as previously described (Green and Sambrook 2012). The blot was blocked in TBST (10 mM Tris-HCl, pH 8.0, 150 mM NaCl, 0.05% Tween20) containing 5% non-fat dry milk for 1 h, washed three times in TBST and incubated in TBST-1% non-fat dry milk containing a primary antibody (prohibitin-2 polyclonal antibody or GFP polyclonal antibody or gp64 monoclonal antibody) with 1:10,000 dilution followed by a secondary antibody (anti-rabbit or anti-mouse IgG antibody for polyclonal and monoclonal primary antibodies, respectively) conjugated with alkaline phosphatase (1:10,000) overnight at room temperature. The blot was washed and developed using nitro blue tetrazolium chloride (NBT) and 5-Bromo-4-chloro-3-indolyl phosphate (BCIP) reagents.

Quantitative RT-PCR

To determine levels of viral DNA accumulation, total genomic DNA was extracted from cells using a genomic DNA extraction kit (Invitrogen) and then subjected to qPCR using specific primers to ie-1 from AcMNPV genome. DNA concentrations were measured by Nanodrop and 10 ng total genomic DNA was used for each qPCR reaction using SYBR Green (Invitrogen) with a Rotor-Gene 6000. Real-Time PCR conditions were 50° C. for 2 min and 95° C. for 2 min followed by 40 cycles of 95° C. for 10 s, 60° C. for 10 s, 72° C. for 20 s, and final extension of 72° C. for 20 s. Transcript levels of GFP, EGFP and prohibitin were analysed by RT-qPCR using gene specific primers, while utilizing the actin and RPS17 genes for insect cells, and HPRT1 gene for Vero and NIH3T3 cells as references (primers shown in Table 4). For each experiment, three biological replicates with three technical replicates were analysed in a Rotor-Gene thermal cycler (QIAGEN) under the following conditions: 95° C. for 30 s, and 40 cycles of 95° C. for 10 s, and 60° C. for 45 s, followed by the melting curve (68° C. to 95° C.). Relative RNA levels from each sample were compared in Qgene template program.

Northern Blot Hybridization

For monitoring dsGFP levels in cells transfected with dsGFP and subsequently infected with AcMNPV, total RNA was extracted from cells at 4, 8, 16, and 24 hpi using Tri-Reagent™ (Molecular Research Center Inc.). Northern blot detection was also carried out by loading 10 μg of total RNA per sample on a 1% agarose gel with 2.2 M formaldehyde. RNA was transferred onto a nylon membrane using 10×SSC (1×SSC is 0.15M NaCl plus 0.015 M sodium citrate). Full-length PCR products of GFP were labelled with [α-³²P]dCTP and used as probe. Hybridization and membrane washes were carried out under high stringency conditions at 65° C. Radioactive signalling was detected by phosphorimaging.

Gene Silencing

To silence genes of interest (GFP, EGFP and prohibitin), we used RNAi by generating dsRNA synthesized in vitro. DNA fragments of ˜500 bp in size were amplified by PCR from the genes of interest. Forward and reverse primers contained T7 promoter sequence at their 5′ end for in vitro RNA synthesis. dsRNA was then produced and purified for each fragment using the MEGAScript kit according to the manufacturer's instructions (Ambion). Synthesis was confirmed by running dsRNA on an agarose gel and the concentration of RNA was determined by measuring absorbance at 260 nm. To induce RNA silencing in vitro, cells were resuspended and equally added to every individual well of a 12-well plate. Once the monolayers had formed (1 h) the medium was removed and a transfection medium was added. This medium consisted of 0.5 ml SF-900 II, 8 μl Cellfectin (Invitrogen), and 2 μg dsRNA either for prohibitin gene or GFP. Twenty-four hours after, each well was infected with 200 μl of AcMNPV inoculum (MOI of 5). The plate was then incubated at 27° C. for 48 h for analyses. For mammalian cells, the procedure was similar except that Lipofectamine was used as a transfection reagent.

### Results

AcMNPV Infection Suppresses the RNAi Response in Sf9 Cells

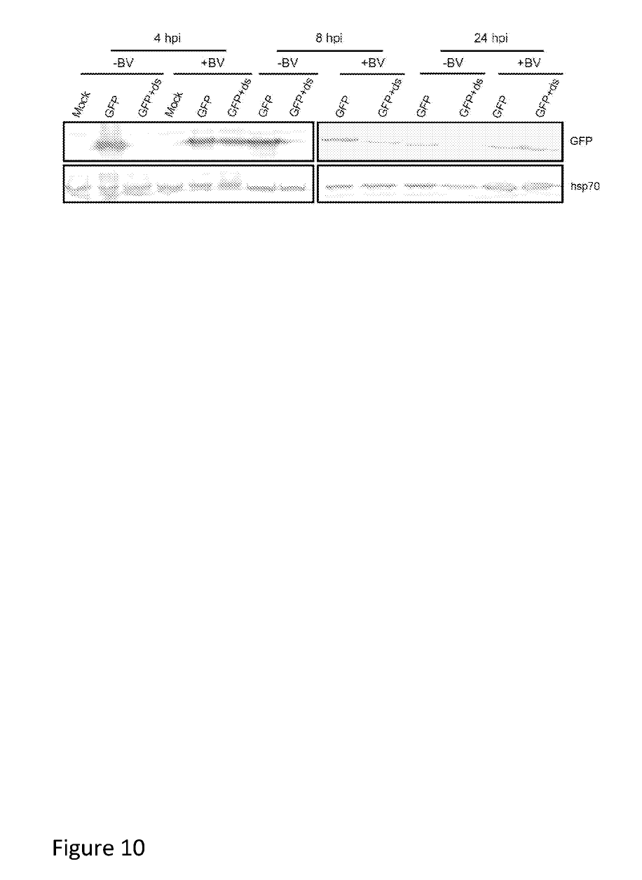

To explore whether AcMNPV suppresses host RNAi, a pIZ/GFP plasmid construct expressing the green fluorescent protein (GFP) gene was co-transfected together with dsRNA GFP (dsGFP) into Sf9 cells, which were subsequently infected with AcMNPV. While GFP was highly expressed in cells transfected with pIZ/GFP only, in cells co-transfected with dsGFP no GFP expression was detected by Western blotting confirming efficient RNAi response in mock-infected Sf9 cells (FIG. 10). Twenty-four hours after co-transfection (pIZ/GFP plus dsGFP), cells were inoculated with AcMNPV and collected at different times post-infection to find out if virus infection impaired the RNAi in the cells. Interestingly, GFP protein was detected in the infected cells in similar quantities in cells transfected with pIZ/GFP only or co-transfected with dsGFP (FIG. 10). These results revealed that AcMNPV infection in Sf9 cells hinders the host RNAi response.

Baculovirus p35 is a Broad Suppressor of RNAi

AcMNPV-mediated suppression of RNAi in Sf9 cells implied that the virus might encode a viral suppressor of RNAi (VSR). To find out the potential gene coding for the VSR, we first queried the virus genome for those genes important in virus-host interactions, particularly, genes responsible for virus defense against host antiviral response. Of those genes, we focused on p35 (ORF-135) due to its prominent anti-apoptosis activity (Clem and Miller 1994). Another reason for this selection was based on a previous study in which an inhibitor of apoptosis (TrAP) from Tomato leaf curl New Delhi virus was shown to have RNAi suppressive activity (Hussain et al. 2007).

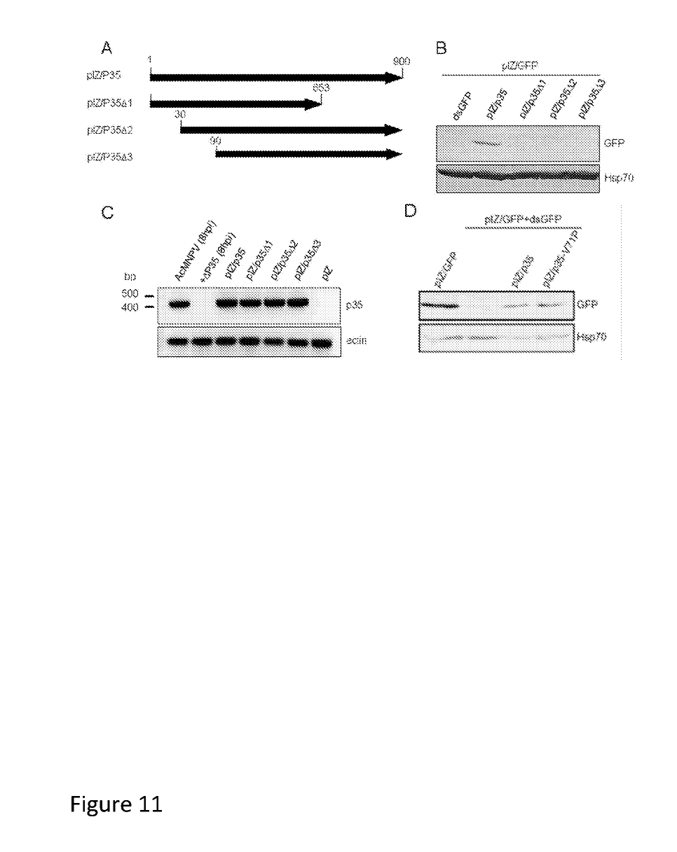

To investigate a potential VSR activity of p35, we inserted the gene coding for the protein into the pIZ/V5 vector (pIZ/p35) and transfected it into Sf9 cells followed by transfection of the cells with pIZ/GFP and dsGFP after 48 h of the initial transfection. Expression of p35 was confirmed by RT-PCR (FIG. 11C). We found that in the presence of pIZ/p35 expression of GFP was restored; in contrast to control cells transfected with pIZ empty vector in which GFP expression was little or close to undetectable (FIG. 12C). This finding suggested that p35 has RNAi suppressor activity. Since GFP was an exogenous marker gene in Sf9 cells, we decided to test if p35 also has suppressor activity in the case of RNAi against an endogenous gene. To examine this, we selected prohibitin, which is a multifunctional endogenous gene, and silenced it in Aedes aegypti Aag2 cells using dsRNA to prohibitin (dsProhibitin). In this case, we also observed that expression of prohibitin, which was suppressed in the presence of dsProhibitin, was rescued in the presence of pIZ/p35 when examined in Western blot detection using polyclonal antibodies to the protein (FIG. 12B). The result was also confirmed at RNA level using RT-qPCR (FIG. 12C).

These results had interesting implications; first, p35 was able to suppress the cellular RNAi machinery against both an exogenous (GFP) and an endogenous (prohibitin) gene, and second, it not only suppressed RNAi in SD cells derived from a lepidopteran, which is a natural and permissive host of AcMNPV, but also in Aag2 cells from a different insect order (Diptera), that is not considered as a host for this virus. To expand this finding further, we analysed GFP transcript levels in Sf9 cells, Drosophila S2 cells, and Helicoverpa zea fat body (HzFB) cells transfected with pIZ/GFP in the presence and absence of pIZ/p35 and dsGFP using RT-qPCR. We consistently observed that p35 displayed RNAi suppressor activity in different cell lines and restored expression of the exogenous gene up to the levels of expression in the control cells (FIG. 12D). Together, the results showed broad RNAi suppressor activity of p35 in different insect cells.

In order to test whether p35 exhibits its RNAi suppressor activity in mammalian cells, we cloned the p35 gene into the pEGFP-N1 vector and used it for transfection of NIH-3T3 mouse cells as well as Vero monkey cells. The EGFP gene in the vector was used as a marker. EGFP expression was efficiently silenced in Vero monkey cells by using dsEGFP (FIG. 4A). However, transcript levels of EGFP after transfection with dsEGFP and pEGFP-N1/P35 were recovered almost to the level of the control cells (FIG. 13A). GATA4 dsRNA was used as control transfection, which did not affect expression of EGFP (FIG. 13A). To further examine the p35 suppressor activity in mammalian cells, transcript levels of prohibitin were monitored in NIH-3T3 mouse cells transfected with dsProhibitin and pEGFP-N1/p35 vector. Likewise, prohibitin expression in NIH-3T3 cells in the presence of dsProhibitin was rescued by p35 to the expression levels of mock-transfected cells (FIG. 13B), while prohibitin transcript levels in cells without p35 expression was significantly reduced (FIG. 13B).

Deletion of p35 Gene from the AcMNPV Genome Abolishes its VSR Activity

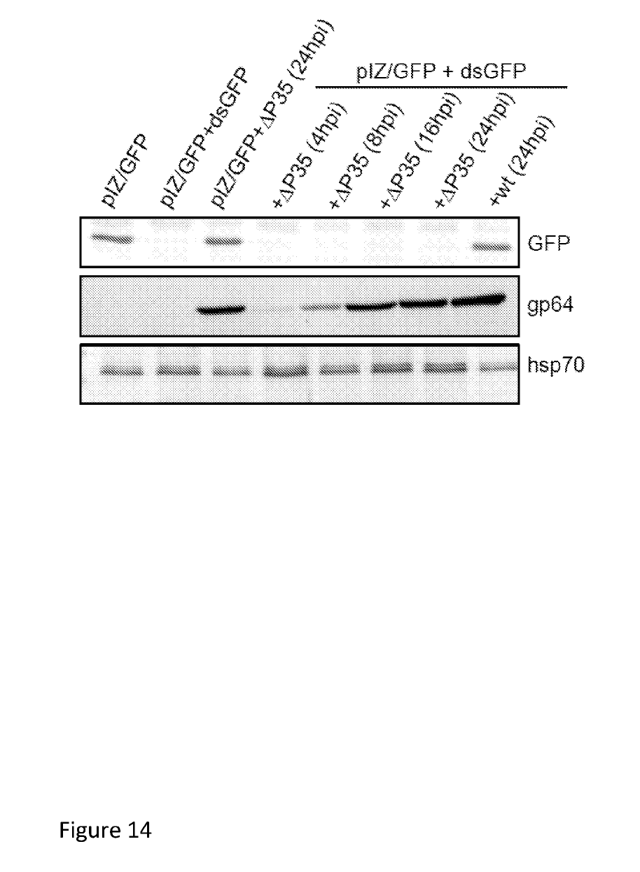

To strengthen our hypothesis that p35 is a VSR of AcMNPV, we used a mutant AcMNPV lacking the p35 gene (Δₚ₃₅AcMNPV) which has previously been extensively characterized. For this, the pIZ/GFP construct and dsGFP were transfected into the Sf9 cells and subsequently infected with the wild type virus and Δₚ₃₅AcMNPV. The lack of p35 expression from this mutant virus was further confirmed by RT-PCR (FIG. 11C). Sf9 cells were then collected at different time intervals post-infection to examine the mutant virus RNAi suppressor activity as compared to the wild type. The results showed that Δₚ₃₅AcMNPV did not suppress the host RNAi as very little to no GFP was detected in the mutant virus-infected cells up to 24 hpi tested, while infection with the wild type AcMNPV impaired host RNAi (FIG. 14). As control, GFP protein was detected in cells transfected with pIZ/GFP only and subsequently infected with the mutant virus. The mutant virus infection and replication was also confirmed by detection of the virus surface protein, gp64 (FIG. 14). The results from this experiment confirmed the RNAi suppressor activity of p35.

p35 Does Not Block Cleavage of dsRNA

To find out if the VSR activity of p35 is due to inhibition of dsRNA degradation by Dicer-2, we first transfected dsGFP into Sf9 cells, and then infected the cells with AcMNPV. Using a specific probe, the presence of dsGFP and its levels in the cells were monitored using Northern blot. We observed that AcMNPV infection did not block dsRNA cleavage, and in fact lower dsGFP levels were found in AcMNPV infected cells as compared with mock-infected Sf9 cells (FIG. 15A). In another experiment, the fate of dsGFP was monitored in Sf9 cells transfected with pIZ/p35 to find out if we detect the same effect observed in AcMNPV-infected cells. Results revealed that p35 does not block cleavage of dsRNA (FIG. 15B). These results suggested that p35 does not display its VSR activity by blocking dsRNA cleavage.

Next, we examined if p35 can still suppress RNAi in the presence of siRNAs. For this, GFP siRNAs (21 nt) were transfected into Sf9 cells for 48 h followed by co-transfection of pIZ/GFP with the empty pIZ vector or pIZ/p35. Western blot analysis showed that siGFP effectively silenced the GFP gene; however, in the presence of pIZ/p35, expression of GFP was rescued (FIG. 15C). This further suggested that p35 blocks RNAi downstream in the RNAi pathway perhaps by sequestering siRNAs, interfering with loading of siRNAs into Ago2 or blocking the activity of Ago2 by protein-protein interaction.

The VSR Activity of p35 is Not Linked to its Anti-Apoptotic Activity

To determine if the VSR activity of p35 is linked to its anti-apoptotic activity, we mutated Valine 71 to proline (p35-Va171P). This single mutation was previously shown to disrupt the spatial configuration of the reactive loop structure in the protein and completely abolish the anti-apoptotic activity of the protein by failing to inhibit the caspase activity (Fisher et al. 1999; dela Crus et al. 2001). The amplified p35-Val71P fragment was cloned into the pIZ vector and the mutation was confirmed by sequencing. Sf9 cells were co-transfected with pIZ-GFP and dsGFP in the presence of pIZ/p35 or pIZ/p35-Val71P. Cells were then analysed on a Western blot using the anti-GFP antibodies. The analysis showed that both p35 and p35-Val71P equally suppressed silencing of GFP (FIG. 11D). These results indicate that the VSR activity of p35 is not linked to its anti-apoptotic activity.

### Conclusions

We functionally assessed RNAi in AcMNPV-infected cells and established that AcMNPV infection suppresses the RNAi response to both dsGFP (an exogenous gene) and dsProhibitin (an endogenous gene). Subsequently, we discovered that the virus gene, p3.5, that is a well-known anti-apoptosis gene, has VSR activity.

Ectopic expression of p35 independent of the virus in various insect host cells using dsRNAs to exogenous and endogenous genes hampered the host RNAi response. The intensity of RNAi suppressor activity in the presence of p35 was almost the same as that detected in AcMNPV-infected cells. To further support our finding, we tested a p35 knockout AcMNPV and found that it could not suppress the host RNAi response. These results together confirmed that p35 is the VSR encoded by AcMNPV. Furthermore, to explore VSR activity of p35 in non-insect cells, the plasmid expressing p35 was transfected into two different mammalian cell lines (Vero and NIH-3T3). In these cells, p35 showed RNAi suppressor activity when an exogenous (EGFP) or an endogenous (prohibitin) gene were targeted by their corresponding dsRNAs. These results demonstrated that p35 is functional in a diverse range of host cells as a potent VSR.

To determine whether the VSR activity of p35 may be due to its anti-apoptotic activity, we assessed a p35-Val71P mutant for which the anti-apoptotic function of the protein has been previously shown to be abolished (Fisher et al. 1999; dela Crus et al. 2001). Our results showed that the p35-Val71P mutant was still able to suppress the RNAi activity indicating that the protein's VSR activity is not related to its anti-apoptotic function.

The results of this study are particularly surprising because dsRNAs have been used for silencing AcMNPV genes (e.g. Huang et al. 2011; Means et al. 2003), which may appear inconsistent with potent VSR activity of p35. However, in those studies, for efficient gene silencing, large amounts of dsRNAs (60-160 μg) were transfected into cells as compared to relatively small amounts of dsRNAs (2 μg) used in this study that were applied to about the same number of cells. Utilization of large quantities of dsRNA may overload the system masking the VSR activity of p35.

### Example 4

### Comparison of the Effect of Wild Type p35 and p35-trunc-Tail Expression on Baculovirus Yield

### Introduction

The work presented in Examples 1 and 2, above, was performed under the assumption that the p35 protein expressed was the wild type p35 protein, comprising the amino acid sequence set forth in SEQ ID NO:1. However, upon review, it was determined that this protein was in fact the protein comprising the amino acid sequence set forth in SEQ ID NO:4, described herein as p35-trunc-tail. As at the time of writing it is unknown as to whether the experiments presented in Example 3 were conducted using wild type p35, or the p35-trunc-tail. Experiments are currently being undertaken to determine this definitively.

The experiments described in this example were conducted in order to determine whether the effect on baculovirus yield presented in Examples 1 and 2 may be unique to p35-trunc-tail, or alternatively if wild type p35 (and potentially other p35 fragments and/or variants, such as p35-trunc) may also exhibit the observed effects on baculovirus yield.

### Materials and Methods

HzAM1cells were transfected with a p35-trunc-tail or wild type p35 encoding plasmid using cellfectin as the tranfecting agent. 25 μg of each plasmid, mixed with 125 μl cellfectin, was added to 5×10⁵ cells/ml in a total of 25 ml culture. The transfection mixture was incubated at 28° C. for 60 h. A control HzAM1 cell culture was kept with similar conditions.

60 hours post transfection, each transfected culture and the control was diluted back to 5×10⁵ cells/ml and infected by 20% fresh-passage 2 budded HearNPV (accession B3K/L7-16/P2). 10 ml of virus was used in total of 50 ml culture. All the infected shakers were counted on 0, 1, 2 and 3 days post infection.

### Results

Results are presented in Table 5. A substantial increase in HearNPV yield as measured by occlusion bodies (OBs) per cell was detected in HzAM1 cells transfected with wild type p35, and HzAM1 cells transfected with p35-trunc-tail, as compared to non-transfected cells. The relative increase in OBs was similar for both wild type p35 and p35-trunc-tail, i.e. the ratio of OBs/cell in wild type p35 transfected cells to non-transfected cells was 1.42, and the ratio of OBs/cell in p35-trunc-tail transfected cells to non-transfected cells was 1.47. Notably, although substantial, the increases in OBs/cell observed for this example were somewhat lower than those observed in Examples 2 and 3. Slower than usual overall growth of all cells lines were observed for this experiment, which appears likely to have contributed to a smaller magnitude of increase in OB yield as compared to that observed in the experiments set forth in Examples 2 and 3 above.

The results of this study confirm that wild type p35 is similarly suitable for expression in host cells to increase baculovirus yield as demonstrated for p35-trunc-tail in Example 1 and 2, above. In view of these results it will be further appreciated that, in addition to p35-trunc-tail (which protein is a fragment and variant of wild type p35, as hereinabove described) at least some other p35 fragments and variants are likely to be suitable for expression in host cells to increase baculovirus yield.

### Example 5

### Expression of p35 in HzAM1 by Infection using AcMNPV

### Introduction

Experiments were conducted to assess whether infection of HzAM1 cells with a p35-encoding virus prior to, or in conjunction with, a baculovirus for which increased yield is sought could increase yield of the baculovirus. For this example, HzAM1 cells were pre-infected with wild type AcMNPV, which encodes p35. Although HzAM1 cells are not natural host cells for AcMNPV, and AcMNPV does not undergo any substantial replication in HzAM1 cells, without being bound by theory the inventors speculate that AcMNPV may express some proteins such as p35 during a partial interaction with HzAM1.

### Material and Methods

HzAM1 cells pre-infected with AcMNPV at a multiplicity of infection of 10 PFU/cell for 5 hours and control HzAM1 cells were diluted to 5×10⁵ cells/ml and infected by 20% fresh-passage 2 budded HearNPV (accession B3K/L7-16/P2). 10 ml of virus was used in total of 50 ml culture. All the infected shakers were counted on 0, 1, 2 and 3 days post infection. These experiments were performed in conjunction with those performed for Example 4, above.

### Results

Results are set forth in Table 6. A substantial increase in HearNPV yield as measured by occlusion bodies (OBs) per cell was detected in HzAM1 cells pre-infected with AcMNPV, as compared to HzAM1 cells which had not been pre-infected. The ratio of OBs/cell detected in AcMNPV pre-infected HzAM1 cells as compared to HzAM1 cells which had not been pre-infected was 1.66.

### Conclusions

This example demonstrates that an increase in baculovirus yield in a host cell may be achieved by pre-infection of the host cell with a virus (such as another baculovirus) which encodes p35. In particular, it will be appreciated that an increase in HearNPV yield HzAM1 cells was achieved by pre-infection of HzAM1 with a AcMNPV wherein HzAM1 cells are not natural host cells for AcMNPV. Without being bound by theory the inventors speculate that, despite not substantially replicating or accumulating in HzAM1, AcMNPV may express p35 in HzAM1 during pre-infection. It is speculated that the presence of p35 in HzAM1 cells pre-infected with AcMNPV resulted in the accumulation of HearNPV observed in this example.

It will be appreciated that pre-infection of a host cell with a wild type virus to engineer the host cell to express one or more of the proteins described herein may be a desirable alternative to transfection of a host cell with a genetic construct to express said proteins in some circumstances. In particular, such a strategy may result in engineered host cells that are considered non-genetically modified, from a regulatory perspective.

### TABLES

TABLE 1
Relative polyhedrin expression in infected, transiently transfected
P35-trunc-tail-HzAM1 cells compared with infected, non-transfected
HzAM1 control cells, derived from SDS PAGE polyhedrin densitometry
studies. Two-tail T test analyses identified the statistical
significance of the difference in the mean polyhedrin level between
the transfected and non-transfected cases.

| | Relative | Mean relative | |
|---|---|---|---|
| | Polyhedrin | Polyhedrin | |
| | expression | expression | Test for |
| | based on | based on | statistical |
| | SDS PAGE | SDS PAGE | significant |
| | densitometry | densitometry | difference |
| Dataset Designation | analysis | analysis | (α: 0.05) |
| Non-transfected control | 1.00 | 1.00 | t(2) = 5.67, |
| HzAM1 R1 (for | | | p(0.0346) < |
| transient) | | | α(0.05), cannot |
| Non-transfected control | 1.00 | | accept null |
| HzAM1 R2 (for | | | hypothesis: |
| transient) | | | Difference is |
| Transiently transfected | 1.49 | 1.60 | statistically |
| P35-trunc-tail- | | | significant |
| HzAM1 R1 | | | |
| Transiently transfected | 1.70 | | |
| P35-trunc-tail- | | | |
| HzAM1 R2 | | | |

TABLE 2
Calculated OB/cell yield from OB counts.

| | Mean | OBs/cell yield range |
|---|---|---|
| Data set designation | OBs/cell | (Cumulative error: ±30%) |
| Non-transfected HzAM1 | 442 | 310-575 |
| (transient control) (a) | | |
| Transiently transfected P35- | 748 | 524-973 |
| trunc-tail-HzAM1(a) | | |
| Non-transfected HzAM1 (stable | 388 | 272-504 |
| control) (b) | | |
| Stably transfected P35-trunc-tail- | 673 | 471-875 |
| HzAM1 (b) | | |

TABLE 3
Ratio in OB/cell yield (p35-trunc-tail HzAM1/HzAM1)
for the Transient and Stable transfection cases.

| Comparison case for transfected p35- | Apparent ratio in OB/cell |
|---|---|
| trunc-tail HzAM1 versus non-transfected | yield (p35-trunc-tail |
| HzAM1 | HzAM1/HzAM1) |
| Transient Transfection | 1.69 |
| Stable Transfection | 1.73 |

TABLE 4
List of primers used for Example 3.

| Primer | sequence |
|---|---|
| GFP-For | CCCAAGCTTCGCCACCATGGTGAGCAA |
| GFP-Rev | CGGGGTACCCTTGTACAGCTCGTCCATGC |
| p35-F SacI | GGGAGCTCATGTGTGTAATTTTTCCGGTAG |
| Δp35-F-SacI | GGGAGCTCATGTAATGTGTAATTTTTCCGGTAG |
| p35-F SacI (-30) | GGGAGCTCATGGTGTCCCAGACGATTATTC |
| p35-F SacI (-90) | GGGAGCTCATGATTAACAAGATTATGAACACGC |
| p35-R SacII | GGCCGCGGTTTATTGTGTTTAATATTAC |
| p35-mid F | CAAAACCCGTTCTCATGATGTT |
| p35-mid R | GTGAGCAAACGGCACAATAAC |
| Ac-orf27-F | GGGAGCTCATGAACGAGGACACGCCCCCG |
| Ac-orf27-R | GGCCGCGGCACCACAAATATTTTTATAAATC |
| Prohibitin-For | ATCCCGGCTACCTGAAGCTG |
| Prohibitin-Rev | GGACATATCGTCGAACTCGG |
| Actin-For | ATGGAGAAGATCTTGCAC |
| Actin-Rev | GGAGCCTCCGTGAGCAGC |
| GFP-RNAi-For | TAATACGACTCACTATAGGGGGTGATGCAACATACGG |
| GFP-RNAi-Rev | TAATACGACTCACTATAGGGGCAGATTGTGTGGACAG |
| Prohibitin RNAi-For | TAATACGACTCACTATAGGGGGCCGGAAAATTCGGCAAGG |
| Prohibitin RNAi-Rev | TAATACGACTCACTATAGGGAATCCTTCGCGCGCTCGACC |
| EGFP-RNAi-For | TAATACGACTCACTATAGGGAGGGCGATGCCACCTACG |
| EGFP-RNAi-Rev | TAATACGACTCACTATAGGGTCAGGGCGGACTGGGTGC |
| EGFP-qPCR-F | TGAGCAAGGGCGAGGAGC |
| EGFP-qPCR-R | TAGGTGGCATCGCCCTCG |
| Mouse-prohib-RNAi-F | TAATACGACTCACTATAGGGATGTGGATGCTGGACACAGA |
| Mouse-prohib-RNAi-R | TAATACGACTCACTATAGGGAGCCACCTGTTTGGCTTCTA |
| Mus-prohib-qPCR-F | GCTGAGCTGATCGCCAACT |
| Mus-prohib-qPCR-R | GGAGAGCTGGTACGCAATGT |
| RPS17-For | CACTCCCAGGTCCGTGGTAT |
| RPS17-Rev | GGACACTTCCGGCACGTAGT |
| HPRT1-For | TGACACTGGCAAAACAATGCA |
| HPRT1-Rev | GGTCCTTTTCACCAGCAAGCT |
| GATA4-RNAi-F | TAATACGACTCACTATAGGGGCAGCATAGCGATAACAGCA |
| GATA4-RNAi-R | TAATACGACTCACTATAGGGAACTGTTGAACGACGGTTCC |

TABLE 5
HearNPV occlusion bodies in wild type p35 transfected HzAM1 cells, p35-
trunc-tail transfected HzAM1 cells, and non-transfected HzAM1 cells.
20% HearNPV

| Name | Replicate | OBs/ml | PTCD/ml | OBs/cell | Average | SE |
|---|---|---|---|---|---|---|
| p35-wild type | R1 | 1.79E+08 | 5.40E+05 | 3.31E+02 | 3.37E+02 | 8.043807 |
| p35 wild | R2 | 1.92E+08 | 5.60E+05 | 3.43E+02 | | |
| type | | | | | | |
| p35-trunc- | R1 | 2.05E+08 | 5.70E+05 | 3.60E+02 | 3.51E+02 | 12.71552 |
| tail | | | | | | |
| p35-trunc- | R2 | 2.05E+08 | 6.00E+05 | 3.42E+02 | | |
| tail | | | | | | |
| HzAM1 | R1 | 1.55E+08 | 6.60E+05 | 2.35E+02 | 2.38E+02 | 4.730528 |
| non- | | | | | | |
| transfected | | | | | | |
| HzAM1 | R2 | 1.57E+08 | 6.50E+05 | 2.42E+02 | | |
| non- | | | | | | |
| transfected | | | | | | |

TABLE 6
HearNPV occlusion bodies in HzAM1 cells pre-infected with AcMNPV.
AcMNPV(10 MOI/HearNPV(20%)/Hzea

| Name | Replicate | OBs/ml | PTCD/ml | OBs/cell | Average | SE |
|---|---|---|---|---|---|---|
| Ac/HNPV/Hz | R1 | 2.40E+08 | 5.80E+05 | 4.14E+02 | 3.94E+02 | 27.43087 |
| Ac/HNPV/Hz | R2 | 2.25E+08 | 6.00E+05 | 3.75E+02 | | |

Throughout the specification, the aim has been to describe the preferred embodiments of the invention without limiting the invention to any one embodiment or specific collection of features. Various changes and modifications may be made to the embodiments described and illustrated without departing from the present invention.

The disclosure of each patent and scientific document, computer program and algorithm referred to in this specification is incorporated by reference in its entirety.

### REFERENCES

Clem R J. 2007. Baculoviruses and apoptosis: A diversity of genes and responses. Curr Drug Targets 8:1069-1074.
Clem R J, Miller L K. 1994. Control of programmed cell death by the baculovirus genes p35 and iap. Mol Cell Biol 14:5212-5222.

dela Cruz W, Friesen P, Fisher A. 2001. Crystal structure of baculovirus P35 reveals a novel conformational change in the reactive site loop after caspase cleavage. J Biol Chem 276:32933-32939.
Fisher A, dela Cruz W, Zoog S, Schneider C, Friesen P. 1999. Crystal structure of baculovirus P35: role of a novel reactive site loop in apoptotic caspase inhibition. EMBO J 18:2031-2039.
Green M R, Sambrook J. 2012. Molecular Cloning: A Laboratory Manual. Molecular cloning. Cold Spring Harbor Laboratory Press.
Herniou E A, Olszewski J A, Cory J S, O'Reilly D R. 2003. The genome sequence and evolution of baculoviruses. Annu Rev Entomol 48:211-234.
Huang N, Wu W, Yang K, Passarelli A, Rohrmann G, Clem R. 2011. Baculovirus infection induces a DNA damage response that is required for efficient viral replication. J Virol 85:12547-12556.
Hussain M, Mansoor S, Iram S, Zafar Y, Briddon R W. 2007. The hypersensitive response to Tomato leaf curl New Delhi virus nuclear shuttle protein is inhibited by transcriptional activator protein. MPMI 20:1581-1588.
Inceoglu A B, Kamita S G, Hammock B D. 2006. Genetically modified baculoviruses: a historical overview and future outlook. Adv Virus Res 68:323-360.
King L A, Possee R D. 1992. The Baculovirus expression system: A laboratory guide. Chapman & Hall.
Means J, Muro I, Clem R. 2003. Silencing of the baculovirus Op-iap3 gene by RNA interference reveals that it is required for prevention of apoptosis during Orgyia pseudotsugata M nucleopolyhedrovirus infection of Ld652Y cells. J Virol 77:4481-4488.
Moscardi F, Souza M L, Castro M E B, Moscardi M, Szewczyk B. 2011. Baculovirus pesticides: present state and future perspectives. In: Ahmad, I., F. Ahmad and J. Pichtel (eds) Microbes and Microbial Technology. Springer, New York, USA, pp: 415-445.

1. A method for increasing or enhancing baculovirus yield in a host cell, wherein said baculovirus does not normally encode a protein comprising the amino acid sequence set forth in SEQ ID NO:1 or a fragment or variant thereof, said method including the step of engineering said host cell to express one or more proteins comprising the amino acid sequences set forth in SEQ ID NOS: 1, 3 or 4, or fragments or variants thereof, to thereby increase or enhance the yield of the baculovirus in the host cell.

2. The method of claim 1, wherein said host cell is engineered to express a protein comprising the amino acid sequence set forth in SEQ ID NO: 1, or a fragment or variant thereof.

3. A method for increasing or enhancing baculovirus yield in a host cell wherein said baculovirus does not normally encode a protein comprising the amino acid sequence set forth in SEQ ID NO: 2 or a fragment or variant thereof, said method including the step of engineering said host cell to express a protein comprising the amino acid sequence set forth in SEQ ID NO: 2 or a fragment or variant thereof, to thereby increase or enhance the yield of the baculovirus in the host cell.

4. A method for increasing or enhancing baculovirus yield in a host cell, said method including engineering said host cell to express a protein comprising the amino acid sequence set forth in SEQ ID NOS: 3 or 4 or a fragment or variant thereof, to thereby increase or enhance baculovirus yield in the host cell.

5. The method of claim 1, wherein said method includes the step of infecting a host cell with a virus to thereby express said one or more proteins in the host cell, wherein the virus with which the host cell is infected to express said one or more proteins in the host cell is a different virus than the baculovirus for which yield is increased or enhanced in the host cell.

6. A method for producing an isolated host cell suitable for infection by a baculovirus, said method including engineering a host cell such that said host cell expresses two or more of the proteins comprising the amino acid sequences set forth in SEQ ID NOS: 1, 3 or 4, or fragments or variants thereof.

7. A method for producing an isolated host cell suitable for infection by a baculovirus, said method including engineering a host cell such that said host cell expresses:
(i) at least one of the proteins comprising the amino acid sequence set forth in SEQ ID NOS: 1, 3 or 4, or a fragment or variant thereof; and
(ii) a protein comprising the amino acid sequence set forth in SEQ ID NO: 2 or a fragment or variant thereof,
to thereby produce the isolated host cell suitable for infection by a baculovirus.

8. (canceled)

9. An isolated, baculovirus-infected host cell, wherein said host cell is engineered to express one or more of the proteins comprising the amino acid sequence set forth in SEQ ID NOS: 1, 3 or 4, respectively, or fragments or variants thereof.

10. The isolated, baculovirus-infected host cell of claim 9, wherein: said baculovirus does not normally encode a protein comprising the amino acid sequence set forth in SEQ ID NO: 1 or a fragment or variant thereof, and the host cell is engineered to express a protein comprising the amino acid sequence set forth in SEQ ID NO: 1, or a fragment or variant thereof; or said baculovirus normally encodes a protein comprising the amino acid sequence set forth in SEQ ID NO: 1 or a fragment or variant thereof.

11. An isolated, baculovirus-infected host cell, wherein said baculovirus does not normally encode a protein comprising the amino acid sequence set forth in SEQ ID NO: 2 or a fragment or variant thereof, and said host cell is engineered to express a protein comprising the amino acid sequence set forth in SEQ ID NO: 2 or a fragment or variant thereof.

12. An isolated, baculovirus-infected host cell, wherein said host cell is engineered to express a protein comprising the amino acid sequence set forth in SEQ ID NOS: 3 or 4, or a fragment or variant thereof.

13. An isolated host cell suitable for infection by a baculovirus, wherein said host cell is capable of expressing two or more of the proteins comprising the amino acid sequence set forth in SEQ ID NOS: 1, 3 or 4, or fragments or variants thereof.

14. An isolated host cell suitable for infection by a baculovirus, wherein said host cell is capable of expressing:
(i) at least one of the proteins comprising the amino acid sequences set forth in SEQ ID NOS: 1, 3 or 4, respectively, or a fragment or variant thereof; and
(ii) a protein comprising the amino acid sequence set forth in SEQ ID NO: 2 or a fragment or variant thereof.

15. (canceled)

16. A method for producing a baculovirus, said method including the step of cultivating the host cell of claim 9 that comprises a baculovirus, to thereby produce the baculovirus.

17. A method for producing a recombinant protein from a baculovirus, said method including the step of cultivating the host cell of claim 9 that comprises a recombinant baculovirus, to thereby produce the recombinant protein from the baculovirus.

18. (canceled)

19. (canceled)

20. An isolated protein consisting essentially of the amino acid sequence set forth in SEQ ID NOS: 3 or 4, or a fragment or variant thereof.

21. An isolated nucleic acid encoding the isolated protein of claim 20.

22. A genetic construct comprising the isolated nucleic acid of claim 21.

23. (canceled)

24. An antibody or antibody fragment that binds or is raised against the isolated protein of claim 20, wherein said antibody or antibody fragment does not bind the amino acid sequence set forth in SEQ ID NO: 1.


SEQUENCE LISTING (9 sequences)

SEQ ID NO: 1 - protein, 299 aa, Autographa californica multiple nucleopolyhedrovirus
  p35 protein from AcMNPV
MCVIFPVEIDVSQTIIRDCQVDKQTRELVYINKIMNTQLTKPVLMMFNISGPIRSVTRKNNNLRDRIKSKVDEQFDQLERDYSDQMDGFHDSIKYFKDEHYSVSCQNGSVLKSKFAKILKSHDYTDKKSIEAYEKYCLPKLVDERNDYYVAVCVLKPGFENGSNQVLSFEYNPIGNKVIVPFAHEINDTGLYEYDVVAYVDSVQFDGEQFEEFVQSLILPSSFKNSEKVLYYNEASKNKSMIYKALEFTTESSWGKSEKYNWKIFCNGFIYDKKSKVLYVKLHNVTSALNKNVILNTIK

SEQ ID NO: 2 - protein, 446 aa, Spodoptera littoralis nucleopolyhedrovirus
  p49 protein from SlNPV
MCVLIPTFNASATTIVDCANLSDSSMRDLIYVNNVAVSKNGNYINRAVIMALNISGPLVCVNRVSMHIVHMYRSHIDRVFDKFNKLTYSATVTDGGGADRVFNSDDYTVVCMSRGDLLNNYKNCLLNEMGATYDDVEKFRKYCLKPLTETENDMMSGSDVGVEDQKKPYVVICSLKPKLLNNKKTLCFTYKPQTGQVIVPLMHEINENGSDVYAYEVMAMIKDVRLSNKPIGVLERLKRSMEQIVLNHNENRYVMTNQLESLNYYLKSINSNKEDNDLNKLMSLLKSLIDQLEKVLHKKSICDFSDDEDDCDVLVDVKYHNAMIEDNIRILLIKMQQYIDDRHMFLPPSLSYENSTKIRLFENVTSFMKCFSLGELYSFVKKIIDWKTHETNQKLACPEVVDDKYAFVKYDYFGTAHGFVYDQRDRTMYVKLHCGIAANRNIYIDI

SEQ ID NO: 3 - protein, 64 aa, Autographa californica multiple nucleopolyhedrovirus
  mutant p35-trunc protein
MCVIFPVEIDVSQTIIRDCQVDKQTRELVYINKIMNTQLTKPVLMMFNISGPIRSVTRKNNNLR

SEQ ID NO: 4 - protein, 71 aa, Autographa californica multiple nucleopolyhedrovirus
  mutant p35-trunc-tail protein
MCVIFPVEIDVSQTIIRDCQVDKQTRELVYINKIMNTQLTKPVLMMFNISGPIRSVTRKNNNLRQNKIKSR

SEQ ID NO: 5 - DNA, 906 bp, Autographa californica multiple nucleopolyhedrovirus
  p35 gene from AcMNPV
atgtgtgtaatttttccggtagaaatcgacgtgtcccagacgattattcgagattgtcaggtggacaaacaaaccagagagttggtgtacattaacaagattatgaacacgcaattgacaaaacccgttctcatgatgtttaacatttcgggtcctatacgaagcgttacgcgcaagaacaacaatttgcgcgacagaataaaatcaaaagtcgatgaacaatttgatcaactagaacgcgattacagcgatcaaatggatggattccacgatagcatcaagtattttaaagatgaacactattcggtaagttgccaaaatggcagcgtgttgaaaagcaagtttgctaaaattttaaagagtcatgattataccgataaaaagtctattgaagcttacgagaaatactgtttgcccaaattggtcgacgaacgcaacgactactacgtggcggtatgcgtgttgaagccgggatttgagaacggcagcaaccaagtgctatctttcgagtacaacccgattggtaacaaagttattgtgccgtttgctcacgaaattaacgacacgggactttacgagtacgacgtcgtagcttacgtggacagtgtgcagtttgatggcgaacaatttgaagagtttgtgcagagtttaatattgccgtcgtcgttcaaaaattcggaaaaggttttatattacaacgaagcgtcgaaaaacaaaagcatgatctacaaggctttagagtttactacagaatcgagctggggcaaatccgaaaagtataattggaaaattttttgtaacggttttatttatgataaaaaatcaaaagtgttgtatgttaaattgcacaatgtaactagtgcactcaacaaaaatgtaatattaaacacaattaaataacccggg

SEQ ID NO: 6 - DNA, 1620 bp, Spodoptera littoralis nucleopolyhedrovirus
  p49 gene from SlNPV
aacataaacgcgtgcggcatgctaaacgagtttgctgcgtcagctgtgctattataaaatgacacattagacgttatctgatacagacgacatacgaacggcaaaatgtgtgtactgataccaacattcaacgcctcggcgaccacgatcgtagattgtgccaatctgagcgatagctcgatgcgcgatctgatctatgtgaacaacgttgcggtgtcaaaaaacggcaattacatcaatcgagctgtgatcatggcgctaaatattagcggacccctcgtgtgcgtgaatcgggtgtccatgcacattgtgcacatgtacagatcgcacatcgatagggtctttgataaattcaacaaattaacatattcggcgaccgtgaccgatggcggtggagccgatcgagtttttaactctgacgactataccgttgtctgcatgtcacgcggagatcttttaaacaattacaaaaattgtttgctcaacgaaatgggagctacgtacgacgacgtcgaaaagtttagaaaatactgtctcaaacctttaactgagaccgaaaatgacatgatgagcggcagcgacgtcggtgtggaagatcaaaaaaaaccgtacgtggtgatatgttcgttgaaaccgaaactgttgaataacaaaaagacattgtgcttcacgtacaaacctcagaccggtcaagtgatcgtgccgcttatgcacgaaattaacgaaaacggaagcgatgtgtatgcgtacgaagtgatggcgatgatcaaagacgtgagactgtcgaacaaaccgatcggtgtgctggaacgattgaagcgttccatggaacagatcgtgttaaatcacaacgagaacagatacgtgatgaccaatcaactcgagtctttgaattattatttaaagtccatcaactctaacaaagaggacaacgatctaaacaaactgatgagtttattgaaaagtctgattgatcaattggaaaaagttttgcataaaaaatctatttgtgacttttccgacgacgaagacgactgcgatgttctcgttgatgtaaagtatcacaatgcaatgatcgaagacaacatacgtatacttttgataaagatgcaacagtacattgatgatcgtcacatgtttttgccgccgtcgctgtcatatgagaatagtacaaaaattcgcctgtttgaaaacgtaacgtcgttcatgaaatgtttcagtcttggagaattgtattcgtttgtaaaaaaaattatcgattggaaaacacacgagaccaatcaaaaacttgcgtgccccgaagtcgtcgacgacaagtacgcttttgtaaaatacgactactttggcaccgcacacggattcgtgtacgatcaaagggatcgaacaatgtacgtaaagttgcattgcggtatagccgcgaatcgtaacatttacatagatatataataattgtatatgaaaattgtatatgaaaataaaaatacttgtgtaaaattaaaatgttttattataaaatgagattcacaaccaatagatttaatcacaacccagtctaaattaattttatcacatcgtctttggaatcgtaaatattatcgatgagatcgttgatgtcagatt

SEQ ID NO: 7 - DNA, 192 bp, Autographa californica multiple nucleopolyhedrovirus
  mutant p35-trunc-encoding open reading frame
atgtgtgtaatttttccggtagaaatcgacgtgtcccagacgattattcgagattgtcaggtggacaaacaaaccagagagttggtgtacattaacaagattatgaacacgcaattgacaaaacccgttctcatgatgtttaacatttcgggtcctatacgaagcgttacgcgcaagaacaacaatttgcga

SEQ ID NO: 8 - DNA, 216 bp, Autographa californica multiple nucleopolyhedrovirus
  mutant p35-trunc-tail-encoding open reading frame
atgtgtgtaatttttccggtagaaatcgacgtgtcccagacgattattcgagattgtcaggtggacaaacaaaccagagagttggtgtacattaacaagattatgaacacgcaattgacaaaacccgttctcatgatgtttaacatttcgggtcctatacgaagcgttacgcgcaagaacaacaatttgcgacagaataaaatcaaaagtcgatga

SEQ ID NO: 9 - DNA, 904 bp, Autographa californica multiple nucleopolyhedrovirus
  mutant p35-trunc-tail gene
atgtgtgtaatttttccggtagaaatcgacgtgtcccagacgattattcgagattgtcaggtggacaaacaaaccagagagttggtgtacattaacaagattatgaacacgcaattgacaaaacccgttctcatgatgtttaacatttcgggtcctatacgaagcgttacgcgcaagaacaacaatttgcgacagaataaaatcaaaagtcgatgaacaatttgatcaactagaacgcgattacagcgatcaaatggatggattccacgatagcatcaagtattttaaagatgaacactattcggtaagttgccaaaatggcagcgtgttgaaaagcaagtttgctaaaattttaaagagtcatgattataccgataaaaagtctattgaagcttacgagaaatactgtttgcccaaattggtcgacgaacgcaacgactactacgtggcggtatgcgtgttgaagccgggatttgagaacggcagcaaccaagtgctatctttcgagtacaacccgattggtaacaaagttattgtgccgtttgctcacgaaattaacgacacgggactttacgagtacgacgtcgtagcttacgtggacagtgtgcagtttgatggcgaacaatttgaagagtttgtgcagagtttaatattgccgtcgtcgttcaaaaattcggaaaaggttttatattacaacgaagcgtcgaaaaacaaaagcatgatctacaaggctttagagtttactacagaatcgagctggggcaaatccgaaaagtataattggaaaattttttgtaacggttttatttatgataaaaaatcaaaagtgttgtatgttaaattgcacaatgtaactagtgcactcaacaaaaatgtaatattaaacacaattaaataacccggg